(19) United States
(12) Patent Application Publication
(10) Pub. No.: US 2007/0142389 A1
(43) Pub. Date: Jun. 21, 2007

(54) PIPERIDINE DERIVATIVES

(75) Inventors: Michelle M. Bruendl, Ann Arbor, MI (US); Keri L. Greene, Canton, MI (US); Rex Allen Jennings, Gregory, MI (US); Scott E. Lazerwith, Ann Arbor, MI (US); Joe Nahra, Canton, MI (US); Patrick Michael O'Brien, Stockbridge, MI (US); Kimberly Suzanne Para, Ann Arbor, MI (US); Susan M. Sheehan, Dexter, MI (US)

Correspondence Address: WARNER-LAMBERT COMPANY, 2800 PLYMOUTH RD, ANN ARBOR, MI, 48105, US

(73) Assignee: PFIZER INC., Ann Arbor, MI (US)

(21) Appl. No.: 11/610,696

(22) Filed: Dec. 14, 2006

Related U.S. Application Data
(60) Provisional application No. 60/751,845, filed on Dec. 20, 2005.

(51) Int. Cl. A61K 31/501; A61K 31/497; A61K 31/4743; A61K 31/454; A61K 31/4545; C07D 401/02; C07D 403/02; C07D 498/02

(52) U.S. Cl. 514/252.03; 514/255.05; 514/301; 514/317; 514/326; 514/318; 544/238; 544/405; 546/194; 546/114; 546/210; 546/236

(57) ABSTRACT

The present invention provides compounds of Formula I: 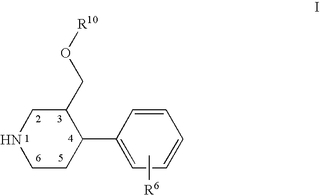
and pharmaceutically acceptable salts thereof, wherein R⁶ and R¹⁰ have any of the values defined therefor in the specification; pharmaceutical compositions; methods of treating conditions, diseases, and disorders; and therapeutic combinations.

### CROSS-REFERENCES TO RELATED APPLICATIONS

This application claims benefit of priority from U.S. Provisional Patent Application No. 60/751,845 filed Dec. 20, 2005.

### BACKGROUND OF THE INVENTION

The monoamines norepinephrine and serotonin have a variety of effects as neurotransmitters. These monoamines are taken up by neurons after being released into the synaptic cleft. Norepinephrine and serotonin are taken up from the synaptic cleft by their respective norepinephrine and serotonin transporters.

Drugs that inhibit the norepinephrine and serotonin transporters can prolong the effects of norepinephrine and serotonin, respectively, in the synapse, providing treatment for a number of diseases. For example, the serotonin reuptake inhibitor fluoxetine has been found to be useful in the treatment of depression and other nervous system disorders. The norepinephrine reuptake inhibitor atomoxetine has been approved for the treatment of attention deficit hyperactivity disorder (ADHD). In addition, the norepinephrine and serotonin transporter inhibitor milnacipran is being developed for the treatment of fibromyalgia, a disease that affects about 2% of the adult population in the United States. However, the FDA has not currently approved any drug for the treatment of fibromyalgia. Accordingly, there is an ongoing need in the art for compounds that are norepinephrine transporter inhibitors, serotonin transporter inhibitors, and that inhibit both norepinephrine and serotonin transporters, for the treatment of diseases including fibromyalgia, ADHD, neuropathic pain, urinary incontinence, generalized anxiety disorder, depression, and schizophrenia.

### SUMMARY OF THE INVENTION

The present invention relates to compounds, and pharmaceutically acceptable salts thereof, pharmaceutical compositions, methods of treatment, and therapeutic combinations. In one aspect, the present invention provides compounds of formula I: 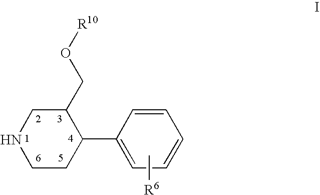
and pharmaceutically acceptable salts thereof, wherein: the positions of the piperidinyl group of Formula I are labeled as 1, 2, 3, 4, 5, or 6; R¹⁰ is 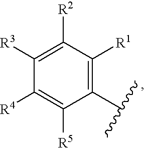
a thienopyridinyl, a 5-membered heteroaryl, or a 6-membered heteroaryl; R¹, R², R³, R⁴, and R⁵ are independently selected from the group consisting of: H; —O—C₅-C₇-cycloalkyl; C₅-C₇-cycloalkyl; —O—C₅-C₇-heterocycloalkyl; -C₅-C₇-heterocycloalkyl; —O—phenyl; phenyl; C₁₋₄alkylene-NR¹⁶R¹⁸; C₁₋₄alkyl; C₁₋₄alkoxy; halo; —C(O)NR¹²R¹⁴; —SO₂—CH₃; —SO₂CH₂CH₃; and CN; wherein said thienopyridinyl, 5-membered heteroaryl or 6-membered heteroaryl may be optionally substituted with one to three substituents independently selected from the group consisting of: H; —O—C₅-C₇-cycloalkyl; C₅-C₇-cycloalkyl; —O—C₅-C₇-heterocycloalkyl; -C₅-C₇-heterocycloalkyl; —O—phenyl;—O—(CH₂)ₙ-phenyl; —O—(CH₂)ₙ-phenyl; phenyl; —O—5- or 6-membered heteroaryl); —O—(CH₂)ₙ-(5- or 6-membered heteroaryl); —H(CH₂)ₙ-(5- or 6-membered heteroaryl); C₁₋₄alkylene-NR¹⁶R¹⁸; C₁₋₄alkyl; C₁₋₄alkoxy; halo; -C(O)NR¹²R¹⁴; —SO₂—CH₃; —SO₂—CH₂CH₃; and CN; n is 1, 2, or 3; and R⁶ is H or one to three substituents independently selected from the group consisting of: C₁₋₄alkyl;
C₁₋₄alkoxy; and halo; provided that when R¹⁰ is 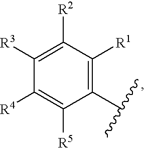
   and R¹, R², R⁴, and R⁵ are H, then R³ is not C₁₋₄alkoxy; and when R¹⁰ is 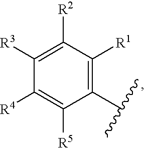
   then one of R¹, R², R³, R⁴, R⁵, and R⁶ is not H.

In certain embodiments of Formula 1, R¹⁰ is 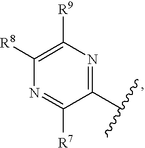
hieno[3,2-b]pyridin-7-yl, 1 H-pyrazol-3-yl, or 2H-pyrazol-3-yl, wherein R⁷, R⁸, and R⁹ are independently selected from the group consisting of: H; —O—C₅-C₇-cycloalkyl; C₅-C₇-cycloalkyl; —O—C₅-C₇-heterocycloalkyl; C₅-C₇-heterocycloalkyl; —O—phenyl; phenyl; C₁₋₄alkylene-NR¹⁶R¹⁸; C₁₋₄alkyl; C₁₋₄alkoxy; halo; -C(O)NR¹²R¹⁴; —SO₂—CH₃; —SO₂—CH₂CH₃; and CN.

In other embodiments, R¹⁰ is 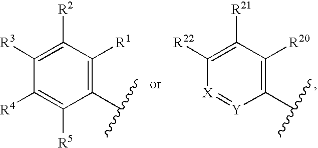
wherein X is C(R²³) and Y is N, or X is N and Y is C(R²⁴); and R²⁰, R²¹, R²², R²³, and R²⁴ are independently selected from the group consisting of: H; —O—C₅-C₇-cycloalkyl; C₅-C₇-cycloalkyl; —O—C₅-C₇-heterocycloalkyl; -C₅-C₇-heterocycloalkyl; —O—phenyl; phenyl; C₁₋₄alkylene-NR¹⁶R¹⁸; C₁₋₄alkyl; C₁₋₄alkoxy; halo; —C(O)NR¹²R¹⁴; —SO₂—CH₃; —SO₂—CH₂CH₃; and CN. In certain embodiments, R¹⁰ is 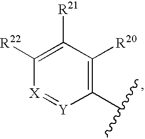
X is C(R²³) and Y is N.

In other embodiments, R¹⁰ is 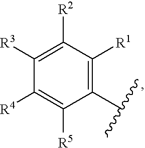
In certain embodiments, R¹, R², R³, R⁴, and R⁵ are independently selected from the group consisting of: H; C₁₋₄alkyl; C₁₋₄alkoxy; halo; —C(O)NR¹²R¹⁴; —SO₂—CH₃; —SO₂—CH₂CH₃; and CN.

In certain embodiments, R¹⁰ is 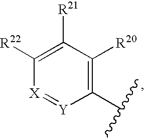
wherein X is N and Y is C(R²⁴). In certain embodiments, R²⁰, R²¹, R²², and R²⁴ are independently selected from the group consisting of: H; C₁₋₄alkyl; C₁₋₄alkoxy; halo; —C(O)NR¹²R¹⁴; —SO₂—CH₃; —SO₂—CH₂CH₃; and CN.

In certain embodiments is a compound selected from the group consisting of:

(3S,4R)-7-(4-phenyl-piperidin-3-ylmethoxy)-thieno[3,2-b]pyridine;

(3S,4R)-3-(2-ethoxy-phenoxymethyl)-4-phenyl-piperidine;

(3S, 4R)-2-(4-phenyl-piperidin-3-ylmethoxy)-benzonitrile;

(3S,4R)-3-(2-fluoro-6-methoxy-phenoxymethyl)-4-phenyl-piperidine;

(3S,4R)-2-ethoxy-3-(4-phenyl-piperidin-3-ylmethoxy)-pyridine;

(3S,4R)-2-methoxy-3-(4-phenyl-piperidin-3-ylmethoxy)-pyridine;

(3S,4R)-3-(4-phenyl-piperidin-3-ylmethoxy)-2-propoxy-pyridine;

(3S,4R)-2-(2-methoxy-ethoxy)-3-(4-phenyl-piperidin-3-ylmethoxy)-pyrid i ne;

(3S,4R)-2-isopropoxy-3-(4-phenyl-piperidin-3-ylmethoxy)-pyridine;

(3S,4R)-2-(3-methoxy-propoxy)-3-(4-phenyl-piperidin-3-ylmethoxy)-pyridine;

(3S,4R)-2-isobutoxy-3-(4-phenyl-piperidin-3-ylmethoxy)-pyridine; and

(±)-trans-2-ethoxy-3-[4-(4-fluoro-phenyl)-piperidin-3-ylmethoxy]-pyridine;

or a pharmaceutically acceptable salt thereof.

In another aspect, the present invention provides for pharmaceutical compositions comprising: a therapeutically effective amount of a compound of Formula I, or a pharmaceutically acceptable salt thereof, and a pharmaceutically acceptable excipient.

In another aspect, the present invention provides for methods of treating a disorder or condition selected from the group consisting of: fibromyalgia; attention deficit hyperactivity disorder (ADHD); generalized anxiety disorder; depression; and schizophrenia, comprising administering to a mammal in need of such treatment a therapeutically effective amount of a compound of formula I, or a pharmaceutically acceptable salt thereof.

The term “alkyl group” or “alkyl” means a monovalent radical of a straight or branched alkane. A “C₁₋₄ alkyl” is an alkyl group having from 1 to 4 carbon atoms. Examples of C₁-C₄ straight—CHain alkyl groups include methyl, ethyl, n-propyl, and n-butyl. Examples of branched—CHain alkyl groups include isopropyl, tert-butyl, isobutyl, etc.

The term alkyl includes both “unsubstituted alkyls” and “substituted alkyls.” Substituted alkyls are alkyl moieties having substituents replacing a hydrogen on one or more carbons of the alkyl. Such substituents are independently selected from the group consisting of: halo; I; Br; Cl; F; trifluoromethyl; —NH₂; —OCF₃; and —O—C₁-C₃ alkyl.

Typical substituted alkyl groups are trifluoromethyl, 2,3-dichloropentyl, 3-hydroxy-5-carboxyhexyl, 2-aminopropyl, pentachlorobutyl, trifluoromethyl, methoxyethyl, 3-hydroxypentyl, 4—CHlorobutyl, 1,2-dimethyl-propyl, and pentafluoroethyl.

The term “C₁-C₄-alkylene” refers to a diradical of an unsubstituted or substituted C₁-C₄-alkane that may be straight or branched chain. Examples of C₁-C₄-alkylene groups include —CH₂-, —CH₂—CH₂-, —CH₂—CH(CH₃)—CH₂-, and -(CH₂)C₁₋₃-. Alkylene groups can be substituted with substituents as described above for alkyl.

“C₁-C₄-alkoxy” refers to a “C₁₋₄ alkyl” group, as defined herein, bound through an oxygen. Examples of unsubstituted C₁-C₄-alkoxy include methoxy, ethoxy, isopropoxy, tert-butoxy, and the like. The term “alkoxy” is intended to include both substituted and unsubstituted alkoxy groups. Alkoxy groups can be substituted on carbon atoms with substituents independently selected from the group consisting of: halo; I; Br; Cl; F; trifluoromethyl; —NH₂; —OCF₃; and —O—C₁-C₃ alkyl. Examples of a C₁-C₄ alkoxy group substituted with a —O—C₁-C₃ alkyl group include methoxyethoxy, methoxy-n-propoxy, etc. Typical substituted alkoxy groups include aminomethoxy, trifluoromethoxy, 2-diethylaminoethoxy, and the like.

“Halo” includes fluoro, chloro, bromo, and iodo.

The term “C₅-C₇cycloalkyl” refers to a monovalent radical of a monocyclic alkane containing from 5 to 7 carbons. Examples of “C₅-C₇cycloalkyl” include cyclopentyl, cyclohexyl, and cycloheptyl. A “C₅-C₇cycloalkyl” may be unsubstituted or substituted with 1 or 2 groups independently selected from the group consisting of: —OH; C₁-C₄alkyl; and —O—C₁-C₄alkyl.

The phrase “5- to 7-membered heterocycloalkyl” means a cyclic group having carbon atoms and 1 or 2 heteroatoms independently selected from the group consisting of: S; N; and O; wherein when two O atoms or one O atom and one S atom are present, the two O atoms or one O atom and one S atom are not bonded to each other, respectively. Illustrative examples of 5- to 7-membered heterocycloalkyls include tetrahydrofuranyl, tetrahydrothienyl, imidazolidinyl, oxazolidinyl, isoxazolidinyl, pyrrolidinyl, tetrahydropyranyl, 1,4-dithianyl, hexahydropyrimidine, morpholinyl, piperazinyl, piperidinyl, tetrahydrothiopyranyl, thiomorpholinyl, azepanyl, oxepanyl, and thiepanyl. The term “heterocycloalkyl” is intended to include both substituted and unsubstituted heterocycloalkyl groups. Heterocycloalkyl groups can be substituted with 1 to 3 groups independently selected from the group consisting of: oxo; C₁₋₄alkyl; and C₁₋₄alkoxy.

A “5-membered heteroaryl” is a monovalent radical of a 5-membered, monocyclic, heteroaromatic ring having from 1 to 4 carbon atoms and from 1 to 4 heteroatoms selected from the group consisting of: 1 O; 1 S; 1 N; 2 N; 3 N; 4 N; 1 S and 1 N; 1 S and 2 N; 1 O and 1 N; and 1 O and 2 N, wherein the maximum number of O is 1, the maximum number of S is 1, and the number of N is 1, 2, 3, or 4, respectively. Examples of 5-membered heteroaryls include furanyl, 2-furanyl, 3-furanyl, imidazolyl, isoxazolyl, isothiazolyl, oxadiazolyl, oxazolyl, pyrazolyl, pyrrolyl, 2- or 3-pyrrolyl, thienyl, 2-thienyl, 3-thienyl, tetrazolyl, thiazolyl, thiadiazolyl, and triazolyl.

A “6-membered heteroaryl” is a monovalent radical of a 6-membered, monocyclic, heteroaromatic ring having from 3 to 5 carbon atoms and from 1 to 3 heteroatoms selected from the group consisting of: 1 N; 2 N; and 3 N, wherein the number of N is 1, 2, or 3, respectively. Examples of 6-membered heteroaryls include pyrazinyl, triazinyl, pyridinyl, pyrimidinyl, pyridin-2-yl, pyridin-4-yl, pyrimidin-2-yl, pyridazin-4-yl, and pyrazin-2-yl. In certain embodiments, the 6-membered heteroaryl has 4 or 5 carbon atoms and 1 or 2 N.

A heteroaryl can also include ring systems substituted on ring carbons with one or more —OH functional groups (which may tautomerize to give a ring C═O group). A heteroaryl can also be substituted on a ring sulfur atom by 1 or 2 oxygen atoms to give S═O, or SO₂ groups, respectively.

“Phenyl” refers to a monovalent radical of benzene. Phenyl groups, unless otherwise noted, may be optionally substituted (i.e., unsubstituted or substituted) with from 1 to 5 substituents independently selected from the group consisting of: C₁-C₄ alkyl; C₁-C₄ alkoxy; halo; OH; —CN; —CF₃; CF₃O—; C₁-C₄ alkyl-S—; phenyl; C₁-C₄ alkyl-C(O)—; C₁-C₄ alkyl-sulfonyl; —C(O)O—R²⁰; —C(O)NR²²R²⁴; and —NR²²R²⁴; where R²⁰ is H or C₁-C₄ alkyl; and R²² and R²⁴ are each independently selected from the group consisting of: H and C₁-C₄ alkyl.

Some of the compounds in the present invention may exist as stereoisomers, including enantiomers, and diastereomers. Some compounds of the present invention have cycloalkyl and/or heterocycloalkyl groups, which may be substituted at more than one carbon atom, in which case all geometric forms thereof, both cis and trans, and mixtures thereof, are within the scope of the present invention. All of these forms, including (R), (S), epimers, diastereomers, cis, trans, syn, anti, solvates (including hydrates), tautomers, and mixtures thereof, are contemplated in the compounds of the present invention.

### DETAILED DESCRIPTION OF THE INVENTION

In one aspect, the present invention provides compounds of formula I, and pharmaceutically acceptable salts thereof. In certain embodiments of Formula I, the 3-position of the piperidinyl group of Formula I is of the R conformation and the 4-position of the piperidinyl group of Formula I is of the S conformation.

In certain embodiments, R¹⁰ is 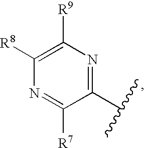
R⁸ and R⁹ are H, and R⁷ is C₁₋₄alkyl, C₁₋₄alkoxy, or halo.

In other embodiments, R¹⁰ is 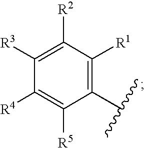
R², R³, and R⁴ are H, and R¹ and R⁵ are independently selected from the group consisting of: C₁₋₄alkyl; C₁₋₄alkoxy; halo; —C(O)NR¹²R¹⁴; —SO₂—CH₃; —SO₂—CH₂CH₃; and CN. In other embodiments, R¹, R², R³, and R⁵ are H, and R⁴ is selected from the group consisting of: C₁₋₄alkyl; C₁₋₄alkoxy; and CN. In other embodiments, R¹, R², R³, and R⁵ are H, and R⁴ is C₁₋₄alkoxy.

In certain embodiments, R¹⁰ is 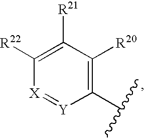
wherein X is N and Y is C(R²⁴); R²¹ and R²² are H, and R²⁰ and R²⁴ are independently selected from the group consisting of: C₁₋₄alkyl; C₁₋₄alkoxy; halo; —C(O)NR¹²R¹⁴; —SO₂—CH₃; —SO₂—CH₂CH₃; and CN. In certain embodiments, R²⁰, R²¹, and R²² are H, and R⁴ is selected from the group consisting of: C₁₋₄alkyl; C₁₋₄alkoxy; and CN. In certain embodiments, R²⁰, R²¹, and R²² are H, and R⁴ is C₁₋₄alkoxy.

### PREPARATION OF COMPOUNDS

Compounds of the present invention (e.g., compounds of Formula I) can be prepared by applying synthetic methodology known in the art and synthetic methodology outlined in the schemes set forth below.

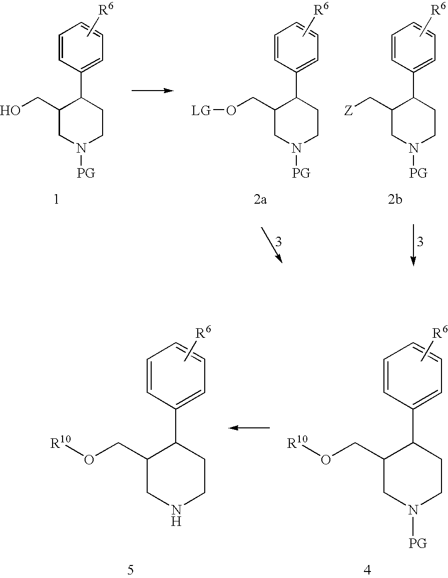

In Scheme 1, the synthesis of a compound of formula 5 is set out. A compound 1 (e.g., (−)-(3S,4R)-3-hydroxymethyl-4-phenyl-piperidine-1-carboxylic acid tert-butyl ester) is reacted with a leaving group reagent LG-X such as methanesulfonyl chloride, in the presence of a base such as triethylamine in a suitable solvent (e.g., chloroform, dichloroethane, CH₂Cl₂, etc.) to provide 2a (e.g., (−)-(3S,4R)-3-methanesulfonyloxymethyl-4-phenyl-piperidine-1-carboxylic acid tert-butyl ester). LG of 2a represents a leaving group. LG can be a leaving group such as toluenesulfonyl, trifluoromethanesulfonyl, acetyl, or trifluoroacetyl, where X is bromine or chlorine.

PG of 1 represents an amino protecting group. Those of skill in the art will recognize that a wide variety of protecting groups can be used as a suitable amino protecting group for PG of 1 (see e.g., Greene and Wuts, Protective Groups in Organic Synthesis, Wiley-Interscience; 3rd edition (1999). Examples of suitable amino protecting groups include esters (tert-butyl ester (BOC), 9-fluorenylmethyl ester (Fmoc), benzyl ester, methyl ester, and allyl ester, etc.); and phenyl and benzyl sulfonyl derivatives (e.g., para-toluenesulfonyl, benzylsulfonyl and phenylsulfonyl). Compounds of formula 1 may be synthesized in a manner similar to that described in Amat et al. (2000) J. Org. Chem. 65: 3074-3084.

3 (R¹⁰-OH) (e.g., a pyridinol such as 2-ethoxy-pyridin-3-ol) that has been treated with a hydride base (e.g., NaH) in a suitable aprotic solvent such as DMF (dimethylformamide) or tetrahydrofuran (THF), may then be reacted with 2a or 2b (where Z is Br or Cl) at a temperature of about 105° C. for 2 hours to 18 hours to provide a compound of formula 4 (e.g., (−)-(3S,4R)-3-(2-ethoxy-pyridin-3-yloxymethyl)-4-phenyl-piperidine-1-carboxylic acid tert-butyl ester).

Alternatively, 2a or 2b can be reacted in an aprotic solvent such as THF, CH₃CN, DMF for 16 to 42 hours at 60-75° C. with 3 (R¹⁰-OH) that has been treated with a hindered tertiary base such as 1,8-diazabicyclo[5.4.0]undec-7-ene (DBU), 1,5-diazabicyclo[4.3.0]non-5-ene (DBN), 7-methyl-1,5,7- triazabicyclo[4.4.0]dec-5-ene (TBD), or a hindered tertiary base resin such as TBD-methyl polystyrene basic resin (Novabiochem®, EMD Biosciences, Inc., San Diego, Calif.) to provide 4. Alternatively, 2 may be reacted with 3 in DMF or THF with cesium carbonate to provide 4.

The protecting group PG can then be removed from 4 to provide a compound of formula 5 (e.g., (−)-(3S,4R)-2-ethoxy-3-(4-phenyl-piperidin-3-ylmethoxy)-pyridine). For example, a tert-butyl ester can be hydrolyzed from (−)-(3S,4R)-3-(2-ethoxy-pyridin-3-yloxymethyl)-4-phenyl-piperidine-1-carboxylic acid tert-butyl ester to provide (−)-(3S,4R)-2-ethoxy-3-(4-phenyl-piperidin-3-ylmethoxy)-pyridine using acids such as HCl or TFA (trifluoroacetic acid).

### PHARMACEUTICALLY ACCEPTABLE SALTS AND SOLVATES

The compounds to be used in the present invention can exist in unsolvated forms as well as solvated forms, including hydrated forms.

The compounds of the present invention (e.g., compounds of Formula I) are capable of further forming both pharmaceutically acceptable salts, including acid addition and/or base salts. Pharmaceutically acceptable salts of the compounds of formula (I) include the acid addition and base salts (including disalts) thereof. Examples of suitable salts can be found for example in Stahl and Wermuth, Handbook of Pharmaceutical Salts: Properties, Selection, and Use, Wiley-VCH, Weinheim, Germany (2002); and Berge et al., “Pharmaceutical Salts,” J. of Pharmaceutical Science, 1977;66:1-19.

Pharmaceutically acceptable acid addition salts of the compounds of Formula I include non-toxic salts derived from inorganic acids such as hydrochloric, nitric, phosphoric, sulfuric, hydrobromic, hydroiodic, phosphorus, and the like, as well as the salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include the acetate, aspartate, benzoate, besylate (benzenesulfonate), bicarbonate/carbonate, bisulfate, caprylate, camsylate (camphor sulfonate), chlorobenzoate, citrate, edisylate (1,2-ethane disulfonate), dihydrogenphosphate, dinitrobenzoate, esylate (ethane sulfonate), fumarate, gluceptate, gluconate, glucuronate, hibenzate, hydrochloride/chloride, hydrobromide/bromide, hydroiodide/iodide, isobutyrate, monohydrogen phosphate, isethionate, D-lactate, L-lactate, malate, maleate, malonate, mandelate, mesylate (methanesulfonate), metaphosphate, methylbenzoate, methylsulfate, 2-napsylate (2-naphthalene sulfonate), nicotinate, nitrate, orotate, oxalate, palmoate, phenylacetate, phosphate, phthalate, propionate, pyrophosphate, pyrosulfate, saccharate, sebacate, stearate, suberate, succinate sulfate, sulfite, D-tartrate, L-tartrate, tosylate (toluene sulfonate), and xinafoate salts, and the like of compounds of Formula I. Also contemplated are the salts of amino acids such as arginate, gluconate, galacturonate, and the like.

Acid addition salts of the basic compounds may be prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents.

Pharmaceutically acceptable base addition salts may be formed with metals or amines, such as alkali and alkaline earth metal hydroxides, or of organic amines. Examples of metals used as cations are aluminum, calcium, magnesium, potassium, sodium, and the like. Examples of suitable amines include arginine, choline, chloroprocaine, N,N′-dibenzylethylenediamine, diethylamine, diethanolamine, diolamine, ethylenediamine (ethane-1,2-diamine), glycine, lysine, meglumine, N-methylglucamine, olamine, procaine (benzathine), and tromethamine.

The base addition salts of acidic compounds may be prepared by contacting the free acid form with a sufficient amount of the desired base to produce the salt in the conventional manner. The free acid form may be regenerated by contacting the salt form with an acid and isolating the free acid in a conventional manner. The free acid forms differ from their respective salt forms somewhat in certain physical properties such as solubility in polar solvents.

### PHARMACEUTICAL COMPOSITIONS AND METHODS OF ADMINISTRATION

This invention also provides for pharmaceutical compositions comprising a therapeutically effective amount of a compound of Formula I, or a pharmaceutically acceptable salt thereof together with a pharmaceutically acceptable excipient. The phrase “pharmaceutical composition” refers to a composition suitable for administration in medical or veterinary use. The phrase “therapeutically effective amount” means an amount of a compound, or a pharmaceutically acceptable salt thereof, sufficient to inhibit, halt, or allow an improvement in the disease being treated when administered alone or in conjunction with another pharmaceutical agent or treatment in a particular subject or subject population. For example in a human or other mammal, a therapeutically effective amount can be determined experimentally in a laboratory or clinical setting, for the particular disease and subject being treated.

Generally, compounds of the present invention will be administered as a formulation in association with one or more pharmaceutically acceptable excipients. The term “excipient” is used herein to describe any ingredient other than the compound(s) of the invention. The choice of excipient will to a large extent depend on factors such as the particular mode of administration, the effect of the excipient on solubility and stability and the nature of the dosage form.

It should be appreciated that determination of proper dosage forms, dosage amounts, and routes of administration is within the level of ordinary skill in the pharmaceutical and medical arts, and is described below.

A compound of the present invention can be formulated as a pharmaceutical composition in the form of a syrup, an elixir, a suspension, a powder, a granule, a tablet, a capsule, a lozenge, a troche, an aqueous solution, a cream, an ointment, a lotion, a gel, a transdermal patch, an emulsion, etc. Preferably, a compound of the present invention will cause a decrease in symptoms or a disease indicia associated with a norepinephrine-mediated and/or serotonin-mediated disorder as measured quantitatively or qualitatively.

For preparing pharmaceutical compositions from the compounds of the present invention, pharmaceutically acceptable excipients can be either solid or liquid. Solid form preparations include powders, tablets, pills, capsules, cachets, suppositories, and dispersible granules. A solid excipient can be one or more substances, which may also act as diluents, flavoring agents, binders, preservatives, tablet disintegrating agents, or an encapsulating material.

In powders, the excipient is a finely divided solid, which is in a mixture with the finely divided active component. In tablets, the active component is mixed with the excipient having the necessary binding properties in suitable proportions and compacted in the shape and size desired.

The powders and tablets contain from 1% to 95% (w/w) of the active compound. In certain embodiments, the active compound ranges from 5% to 70% (w/w). Suitable excipients are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a excipient providing a capsule in which the active component with or without other excipients, is surrounded by a excipient, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 1000 mg, preferably 1.0 mg to 100 mg, or from 1% to 95% (w/w) of a unit dose, according to the particular application and the potency of the active component. The composition can, if desired, also contain other compatible therapeutic agents.

Pharmaceutically acceptable excipients are determined in part by the particular composition being administered, as well as by the particular method used to administer the composition. Accordingly, there are a wide variety of suitable formulations of pharmaceutical compositions of the present invention (see, e.g., Remington: The Science and Practice of Pharmacy, 20th ed., Gennaro et al. Eds., Lippincott Williams and Wilkins, 2000).

A compound of the present invention, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane nitrogen, and the like.

Formulations suitable for parenteral administration, such as, for example, by intravenous, intramuscular, intradermal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives. In the practice of the present invention, compositions can be administered, for example, by intravenous infusion, orally, topically, intraperitoneally, intravesically or intrathecally. The formulations of compounds can be presented in unit-dose or multi-dose sealed containers, such as ampules and vials. Injection solutions and suspensions can be prepared from sterile powders, granules, and tablets of the kind previously described.

The dose administered to a subject, in the context of the present invention should be sufficient to affect a beneficial therapeutic response in the subject over time. The term “subject” refers to a member of the class Mammalia. Examples of mammals include, without limitation, humans, primates, chimpanzees, rodents, mice, rats, rabbits, horses, livestock, dogs, cats, sheep, and cows. In certain embodiments, the “subject” is a human.

The dose will be determined by the efficacy of the particular compound employed and the condition of the subject, as well as the body weight or surface area of the subject to be treated. The size of the dose also will be determined by the existence, nature, and extent of any adverse side effects that accompany the administration of a particular compound in a particular subject. In determining the effective amount of the compound to be administered in the treatment or prophylaxis of the disease being treated, the physician can evaluate factors such as the circulating plasma levels of the compound, compound toxicities, and/or the progression of the disease, etc. In general, the dose equivalent of a compound is from about 1 μg/kg to 100 mg/kg for a typical subject. Many different administration methods are known to those of skill in the art.

For administration, compounds of the present invention can be administered at a rate determined by factors that can include the pharmacokinetic profile of the compound, contraindicated drugs, and the side effects of the compound at various concentrations, as applied to the mass and overall health of the subject. Administration can be accomplished via single or divided doses.

An example of a tablet includes the following:

TABLET FORMULATION EXAMPLE 1
Tablet Formulation

| Ingredient | Amount | |
|---|---|---|
| A compound of Formula I | 50 | mg |
| Lactose | 80 | mg |
| Cornstarch (for mix) | 10 | mg |
| Cornstarch (for paste) | 8 | mg |
| Magnesium Stearate (1%) | 2 | mg |
| | 150 | mg |

The compounds of the present invention (e.g., a compound of Formula I, or a pharmaceutically acceptable salt thereof) can be mixed with the lactose and cornstarch (for mix) and blended to uniformity to a powder. The cornstarch (for paste) is suspended in 6 mL of water and heated with stirring to form a paste. The paste is added to the mixed powder, and the mixture is granulated. The wet granules are passed through a No. 8 hard screen and dried at 50° C. The mixture is lubricated with 1% magnesium stearate and compressed into a tablet. The tablets are administered to a patient at the rate of 1 to 4 each day for treatment of a norepinephrine-mediated and/or serotonin-mediated disorder.

### TREATING NOREPINEPHRINE- AND/OR SEROTONIN-MEDIATED DISORDERS

The compounds of the present invention and pharmaceutical compositions comprising a compound of the present invention can be administered to treat a subject suffering from a norepinephrine-mediated and/or serotonin-mediated disorder, including central nervous disorders, which is alleviated by the inhibition of a norepinephrine transporters and/or serotonin transporters.

Norepinephrine-mediated and/or serotonin-mediated disorders can be treated prophylactically, acutely and chronically using compounds of the present invention, depending on the nature of the disease. Typically, the subject in each of these methods is human, although other mammals can also benefit from the administration of a compound of the present invention.

In therapeutic applications, the compounds of the present invention can be prepared and administered in a wide variety of oral and parenteral dosage forms. The term “administering” refers to the method of contacting a compound with a subject. Thus, the compounds of the present invention can be administered by injection, that is, intravenously, intramuscularly, intracutaneously, subcutaneously, intraduodenally, parentally, or intraperitoneally. Also, the compounds described herein can be administered by inhalation, for example, intranasally. Additionally, the compounds of the present invention can be administered transdermally, topically and via implantation. In certain embodiments, the compounds of the present invention are delivered orally. The compounds can also be delivered rectally, bucally, intravaginally, ocularly, or by insufflation.

The compounds utilized in the pharmaceutical method of the invention can be administered at a dosage of about 0.001 mg/kg to about 100 mg/kg daily. In certain embodiments, the daily dose range is from about 0.1 mg/kg to about 10 mg/kg.

The dose administered to a subject, in the context of the present invention should be sufficient to affect a beneficial therapeutic response in the subject over time. The term “subject” refers to a member of the class Mammalia. Examples of mammals include, without limitation, humans, primates, chimpanzees, rodents, mice, rats, rabbits, horses, livestock, dogs, cats, sheep and cows.

Determination of the proper dosage for a particular situation is within the skill of the practitioner. The dose will be determined by the efficacy of the particular compound employed and the condition of the subject, the severity of the disease being treated, as well as the body weight or surface area of the subject to be treated. The size of the dose also will be determined by the existence, nature and extent of any adverse side-effects that accompany the administration of a particular compound in a particular subject. In determining the effective amount of the compound to be administered in the treatment or prophylaxis of the disease being treated, the physician can evaluate factors such as the circulating plasma levels of the compound, compound toxicities, and/or the progression of the disease, etc. In addition, compounds of the present invention can be administered at a rate determined by factors that can include the pharmacokinetic profile of the compound, contraindicated drugs and the side-effects of the compound at various concentrations, as applied to the mass and overall health of the subject.

Generally, treatment is initiated with smaller dosages, which are less than the optimum dose of the compound. Thereafter, the dosage is increased by small increments until the optimum effect under circumstances is reached. For convenience, the total daily dosage may be divided and administered in portions during the day, if desired. The term “treatment” includes the acute, chronic, or prophylactic diminishment or alleviation of at least one symptom or characteristic associated with or caused by the disease being treated. For example, treatment can include diminishment of several symptoms of a disease, inhibition of the pathological progression of a disease, or complete eradication of a disease.

The present invention also relates to a method of treating a norepinephrine-mediated and/or serotonin-mediated disorder, the method comprising administering to a mammal in need of such treatment a therapeutically effective amount of a compound of formula I. Examples of norepinephrine-mediated and/or serotonin-mediated disorders include fibromyalgia, single episodic or recurrent major depressive disorders, dysthymic disorders, depressive neurosis and neurotic depression, melancholic depression including anorexia, weight loss, insomnia, early morning waking or psychomotor retardation; atypical depression (or reactive depression) including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; attention deficit hyperactivity disorder (ADHD); disruptive behavior disorder; behavioral disturbances associated with mental retardation, autistic disorder and conduct disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder and generalized anxiety disorders; borderline personality disorder; schizophrenia and other psychotic disorders, for example, schizophreniform disorders, schizoaffective disorders, delusional disorders, brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; delirium, dementia, and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, and other dementias, for example, due to HIV disease, head trauma, Parkinson's disease, Huntington's disease, Pick's disease, Creutzfeldt-Jakob disease, or due to multiple etiologies; movement disorders such as akinesias, dyskinesias, including familial paroxysmal dyskinesias, spasticities, Tourette's syndrome, Scott syndrome, palsys (e.g., Bell's palsy, cerebral palsy, birth palsy, brachial palsy, wasting palsy, ischemic palsy, progressive bulbar palsy and other palsys), and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremor; chemical dependencies and addictions (e.g., dependencies on, or addictions to, alcohol, heroin, cocaine, benzodiazepines, nicotine, or phenobarbitol) and behavioral addictions such as an addiction to gambling; and ocular disorders such as glaucoma and ischemic retinopathy.

In one particular embodiment, patients suffering from fibromyalgia are administered a therapeutically effective amount of a compound of formula I, or a pharmaceutically acceptable salt thereof. Patients suffering from fibromyalgia typically exhibit a history of widespread pain, and the presence of pain at 11 out of 18 points upon palpatation (see e.g., Wolfe et al. (1990) Arthritis Rheum. 33:160-172). Fibromyalgia patients generally display pain perception abnormalities in the form of both allodynia (pain from innocuous stimulation) and hyperalgesia (an increased sensitivity to a painful stimulation).

Fibromyalgia patients typically also exhibit a range of other symptoms, including sleep disturbance and fatigue. Although less common than pain, fatigue, and sleep problems, a variety of other symptoms may occur as well. These include headaches, morning stiffness, difficulty concentrating, a circulatory problem that affects the small blood vessels of the skin (Raynaud's phenomenon), and irritable bowel syndrome. As with many conditions that cause chronic pain, anxiety and depression are common in fibromyalgia patients and may make symptoms worse. Fibromyalgia symptoms may tend to come and go. There can be periods when the symptoms are constant (flares), which may be followed by periods when the symptoms are absent (remissions). Some fibromyalgia patients find that cold, damp weather, emotional stress, overexertion, and other factors exacerbate their symptoms.

In another aspect, the present invention provides for methods of treating a disorder or condition selected from the group consisting of: urinary incontinence; genuine stress incontinence (GSI); stress urinary incontinence (SUI); urinary incontinence in the elderly; overactive bladder (OAB), which includes OAB due to idiopathic detrusor instability, OAB due to detrusor overactivity secondary to neurological diseases (e.g. Parkinson's disease, multiple sclerosis, spinal cord injury and stroke), and OAB due to detrusor overactivity secondary to bladder outflow obstruction (e.g. benign prostatic hyperplasia (BPH), urethral stricture, or stenosis); nocturnal eneuresis; urinary incontinence due to a combination of the above conditions (e.g. genuine stress incontinence associated with overactive bladder); urinary symptoms, which include urinary frequency and urinary urgency, the methods comprising administering to a mammal in need of such treatment a therapeutically effective amount of a compound of formula I, or a pharmaceutically acceptable salt thereof.

A more specific embodiment of the present invention relates to the above method wherein the disorder or condition that is being treated is selected from the group consisting of: major depression; single episode depression; recurrent depression; child abuse induced depression; postpartum depression; dysthymia; cyclothymia; and bipolar disorder.

Another more specific embodiment of the present invention relates to the above method wherein the disorder or condition that is being treated is selected from the group consisting of: schizophrenia; schizoaffective disorder; delusional disorder; substance-induced psychotic disorder; brief psychotic disorder; shared psychotic disorder; psychotic disorder due to a general medical condition; and schizophreniform disorder.

Another more specific embodiment of the present invention relates to the above method wherein the disorder or condition that is being treated is selected from the group consisting of: autism; pervasive development disorder; and attention deficit hyperactivity disorder.

Another more specific embodiment of the present invention relates to the above method wherein the disorder or condition that is being treated is selected from the group consisting of: generalized anxiety disorder; panic disorder; obsessive-compulsive disorder; post-traumatic stress disorder; and phobias, which include social phobia, agoraphobia, and specific phobias.

Another more specific embodiment of the present invention relates to the above method wherein the disorder or condition that is being treated is selected from the group consisting of: movement disorders; movement disorders that are akinesias; movement disorders that are dyskinesias, which include familial paroxysmal dyskinesias; movement disorders that are spasticities; Tourette's syndrome; Scott syndrome; palsys, which include Bell's palsy, cerebral palsy, birth palsy, brachial palsy, wasting palsy, ischemic palsy, progressive bulbar palsy, and other palsys; akinetic-rigid syndrome; and extra-pyramidal movement disorders, which include medication-induced movement disorders, wherein medication-induced movement disorders include neuroleptic-induced Parkinsonism, neuroleptic malignant syndrome, neuroleptic-induced acute dystonia, neuroleptic-induced acute akathisia, neuroleptic-induced tardive dyskinesia, and medication-induced postural tremor.

Another more specific embodiment of the present invention relates to the above method wherein the disorder or condition that is being treated is pain. Pain refers to acute as well as chronic pain. Acute pain is usually short-lived and is associated with hyperactivity of the sympathetic nervous system. Examples are postoperative pain and allodynia. Chronic pain is usually defined as pain persisting for a minimum of from 3 to 6 months and includes somatogenic pain and psychogenic pain. Other pain is nociceptive.

Examples of the types of pain that can be treated with the compounds of formula I of the present invention and their pharmaceutically acceptable salts (treatable pain) are provided herein. Treatable pain includes pain resulting from soft tissue or peripheral damage such as acute trauma, pain associated with osteoarthritis, pain associated with rheumatoid arthritis; musculo-skeletal pain such as pain experienced after trauma, spinal pain, dental pain, myofascial pain syndromes, episiotomy pain, and pain resulting from burns; deep and visceral pain such as heart pain, muscle pain, eye pain, orofacial pain, for example, odontalgia, abdominal pain, gynaecological pain, for example, dysmenorrhoea, labor pain, and pain associated with endometriosis; pain associated with nerve or root damage such as pain associated with peripheral nerve disorders, for example, nerve entrapment or brachial plexus avulsions, amputation, peripheral neuropathies, tic douloureux, atypical facial pain, nerve root damage, trigeminal neuralgia, neuropathic lower back pain, HIV related neuropathic pain, cancer related neuropathic pain, diabetic neuropathic pain, and arachnoiditis; neuropathic and non-neuropathic pain associated with carcinoma, often referred to as cancer pain; central nervous system pain such as pain due to spinal cord or brain stem damage; lower back pain; sciatica; phantom limb pain; headache, including migraine and other vascular headaches, acute or chronic tension headache, cluster headache, temperomandibular pain, and maxillary sinus pain; pain resulting from ankylosing spondylitis or gout; pain caused by increased bladder contractions; post operative pain; scar pain; and chronic non-neuropathic pain such as pain associated with fibromyalgia, HIV, rheumatoid arthritis or osteoarthritis, arthralgia or myalgia, sprains, strains or trauma such as broken bones; and post surgical pain.

Still other treatable pain is caused by injury or infection of peripheral sensory nerves. Such pain includes, but is not limited to, pain from: peripheral nerve trauma, herpes virus infection, diabetes mellitus, fibromyalgia, causalgia, plexus avulsion, neuroma, limb amputation, or vasculitis. Also included is neuropathic pain, which may be caused by nerve damage from chronic alcoholism, human immunodeficiency virus infection, hypothyroidism, uremia, or vitamin deficiencies. Treatable neuropathic pain includes, but is not limited to pain caused by nerve injury such as, for example, the diabetic neuropathic pain.

Psychogenic pain is treatable and is pain which occurs without an organic origin. Examples of such pain are low back pain, atypical facial pain, and chronic headache.

Examples of other types of treatable pain are inflammatory pain, osteoarthritic pain, trigeminal neuralgia, cancer pain, diabetic neuropathy, restless leg syndrome, acute herpetic neuralgia, postherpetic neuralgia, causalgia, pain of brachial plexus avulsion, occipital neuralgia, gout pain, phantom limb pain, burn pain, other forms of neuralgia, and neuropathic and idiopathic pain syndromes.

Another more specific embodiment of the present invention relates to the above method wherein the disorder or condition that is being treated is selected from the group consisting of: delirium; dementia; and amnestic and other cognitive or neurodegenerative disorders such as Parkinson's disease (PD), Huntington's disease (HD), Alzheimer's disease, senile dementia, dementia of the Alzheimer's type, memory disorders, loss of executive function, vascular dementia, and other dementias due to, for example, HIV disease, head trauma, Parkinson's disease, Huntington's disease, Pick's disease, or Creutzfeldt-Jakob disease, or due to multiple etiologies.

The compounds of the present invention can be co-administered to a subject. The term “co-administered” means the administration of two or more different pharmaceutical agents or treatments (e.g., radiation treatment) that are administered to a subject by combination in the same pharmaceutical composition or separate pharmaceutical compositions. Thus co-administration involves administration at the same time of a single pharmaceutical composition comprising two or more pharmaceutical agents or administration of two or more different compositions to the same subject at the same or different times. For example, a subject that is administered a first dosage that comprises a compound of the present invention at 8 a.m. and then is administered a second therapeutic agent at 1 to 12 hours later, e.g., 6 p.m., of that same day has been co-administered with a compound of the present invention and the second therapeutic agent. Alternatively, for example, a subject could be administered with a single dosage comprising a compound of the present invention and a second therapeutic agent at 8 a.m. has been co-administered with a compound of the present invention and the second therapeutic agent.

The compounds of the present invention may further be co-administered for the treatment of fibromyalgia with one or more agents useful for treating one or more indicia of fibromyalgia. Such agents include non-steroidal anti-inflammatory agents (hereinafter NSAIDs) such as piroxicam, loxoprofen, diclofenac, propionic acids such as naproxen, flurbiprofen, fenoprofen, ketoprofen and ibuprofen, ketorolac, nimesulide, acetominophen, fenamates such as mefenamic acid, indomethacin, sulindac, apazone, pyrazolones such as phenylbutazone, salicylates such as aspirin, COX-2 inhibitors such as celecoxib and etoricoxib; steroids, cortisone, prednisone, muscle relaxants including cyclobenzaprine and tizanidine; hydrocodone, dextropropoxyphene, lidocaine, opioids, morphine, fentanyl, tramadol, codeine, paroxetine, diazepam, femoxetine, carbamazepine, milnacipran, reboxetine, venlafaxine, duloxetine, topisetron, Interferon alpha, cyclobenzaprine, CPE-215, sodium oxbate, citalopram HBr, sertraline HCl, antidepressants, tricyclic antidepressants, amitryptyline, fluoxetine, topiramate, escitalopram, benzodiazepenes including diazepam, bromazepam and tetrazepam, mianserin, clomipramine, imipramine, topiramate, and nortriptyline. The compound of the present invention may also be co-administered with alpha-2-delta ligands. Examples of alpha-2-delta ligands for use with the present invention are those compounds generally or specifically disclosed in U.S. Pat. No. 4,024,175, particularly gabapentin; EP641330 and U.S. Pat. No. 6,197,819, particularly pregabalin; U.S. Pat. No. 5,563,175 and WO9733858, WO9733859, WO9931057, WO9931074, WO97291 01, and WO02085839, particularly [(1R,5R,6S)-6-(aminomethyl)bicyclo[3.2.0]hept-6-yl]acetic acid; WO9931075, particularly 3-(1 -aminomethyl-cyclohexylmethyl)-4H-[1,2,4]oxadiazol-5-one and C-[1-(1 H-tetrazol-5-ylmethyl)-cycloheptyl]-methylamine; WO9921824, particularly (3S,4S)-(1 -aminomethyl-3,4-dimethyl-cyclopentyl)-acetic acid; WO01 90052 and WO01 28978, particularly (1α,3α,5α)(3-amino-methyl-bicyclo[3.2.0]hept-3-yl)-acetic acid; EP0641330, WO9817627, and WO0076958, particularly (3S,5R)-3-aminomethyl-5-methyl-octanoic acid; WO2003/082807, particularly (3S,5R)-3-amino-5-methyl-heptanoic acid, (3S,5R)-3-amino-5-methyl-nonanoic acid, and (3S,5R)-3-Amino-5-methyl-octanoic acid; EP1178034, EP1201240, WO9931074, WO03000642, WO0222568, WO0230871, WO0230881, WO02100392, WO02100347, WO0242414, WO0232736, and WO0228881; and pharmaceutically acceptable salts and solvates thereof, all of which are incorporated herein by reference.

For the treatment of depression, anxiety, schizophrenia, etc., the compounds of the present invention can be used in conjunction with one or more other antidepressants or anti-anxiety agents. Examples of classes of antidepressants that can be used in combination with the active compounds of the present invention include norepinephrine reuptake inhibitors, selective serotonin reuptake inhibitors (SRIs), neurokinin-1 (NK-1) receptor antagonists, monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists, alpha-2-delta ligands (A2D) such as gabapentin and pregabalin, [(1R,5R,6S)-6-(aminomethyl)bicyclo[3.2.0]hept-6-yl]acetic acid, 3-(1-aminomethyl-cyclohexylmethyl)-4H-[1,2,4]oxadiazol-5-one and C-[1-(1 H-tetrazol-5-ylmethyl )-cycloheptyl]-methylamine, (3S,4S)-(1 -aminomethyl-3,4-dimethyl-cyclopentyl)-acetic acid, (1α,3α,5α)(3-amino-methyl-bicyclo[3.2.0]hept-3yl)acetic acid, (3S,5R)-3-aminomethyl-5-methyl-octanoic acid, (3S,5R)-3-amino-5-methyl-heptanoic acid, (3S,5R)-3-amino-5-methyl-nonanoic acid and (3S,5R)-3-amino-5-methyl-octanoic acid, etc.), and atypical antidepressants. Suitable norepinephrine reuptake inhibitors include tertiary amine tricyclics and secondary amine tricyclics. Suitable tertiary amine tricyclics and secondary amine tricyclics include amitriptyline, clomipramine, doxepin, imipramine, trimipramine, dothiepin, butripyline, iprindole, lofepramine, nortriptyline, protriptyline, amoxapine, desipramine and maprotiline. Suitable selective serotonin reuptake inhibitors include fluoxetine, fluvoxamine, paroxetine, citalopram, and sertraline. Examples of monoamine oxidase inhibitors include isocarboxazid, phenelzine, and tranylcyclopramine. Suitable reversible inhibitors of monoamine oxidase include moclobemide. Suitable serotonin and noradrenaline reuptake inhibitors for use in the present invention include venlafaxine and duloxetine. Suitable CRF antagonists include those compounds described in International Patent Application Nos. WO 94/13643, WO 94/13644, WO 94/13661, WO 94/13676 and WO 94/13677. Suitable atypical anti-depressants include bupropion, lithium, nefazodone, trazodone and viloxazine. Suitable NK-1 receptor antagonists include those referred to in World Patent Publication WO 01/77100. Suitable A2D ligands include those referred to in World Patent Publications WO 99/21824, WO 01/90052, WO 01/28978, WO 98/17627, WO 00/76958, and WO 03/082807, and specifically gabapentin and pregabalin.

Suitable classes of anti-anxiety agents that can be used in combination with the active compounds of the present invention include benzodiazepines and serotonin IA (5-HT_{IA}) agonists or antagonists, especially 5-HT_{IA} partial agonists, and CRF antagonists. Suitable benzodiazepines include alprazolam, chlordiazepoxide, clonazepam, chlorazepate, diazepam, halazepam, lorazepam, oxazepam, and prazepam. Suitable 5-HT_{IA} receptor agonists or antagonists include buspirone, flesinoxan, gepirone and ipsapirone.

Suitable antipsychotic agents include both conventional and atypical anti psychotics.

Conventional antipsychotics are antagonists of dopamine (D₂) receptors. The atypical antipsychotics also have D₂ antagonistic properties but possess different binding kinetics to these receptors and activity at other receptors, particularly 5-HT2A, 5-HT_{2C} and 5-HT_{2D} (Schmidt B et al, Soc. Neurosci. Abstr. 24:2177, 1998).

The class of atypical antipsychotics includes clozapine, 8—CHloro-11-(4-methyl-1-piperazinyl)-5H-dibenzo[b,e][1,4]diazepine (U.S. Pat. No. 3,539,573); risperidone, 3-[2-[4-(6-fluoro-1,2-benzisoxazol-3-yl)piperidino]ethyl]-2-methyl-6,7,8,9,tetrahydro-4H-pyrido-[1,2-a]pyrimidin-4-one (U.S. Pat. No. 4,804,663); olanzapine, 2-methyl-4-(4-methyl-1-piperazinyl)-10 thieno[2,3-b][1,5]benzodiazepine (U.S. Pat. No. 5,229,382); quetiapine, 5-[2-(4-dibenzo[b,f][1,4]thiazepin-1 1 -yl-1 -piperazinyl)ethoxy]ethanol (U.S. Pat. No. 4,879,288); aripiprazole, 7-{4-[4-(2,3-dichlorophenyl)-1-piperazinyl]-butoxy}-3,4-dihydro carbostyril and 7-{4-[4-(2,3-dichlorophenyl)-1-piperazinyl]-butoxy}-3,4-dihydro2(1 H)-quinolinone (U.S. Pat. Nos. 4,734,416 and 5,006,528); sertindole, 1-[2-[4-[5—CHloro-1 -(4-fluorophenyl)-1 H-indol-3-yl]-1- piperidinyl]ethyl]imidazolidin-2-one (U.S. Pat. No. 4,710,500); amisulpride (U.S. Pat. No. 4,410,822); and ziprasidone, 5-[2-[4-(1,2-benzisothiazol-3-yl)piperazin-3-yl]ethyl]-6—CHloroindolin-2-one hydrochloride hydrate (U.S. Pat. No. 4,831,031).

### EXAMPLES

Intermediate 1: (−)-(3S,4R)-3-Methanesulfonyloxymethyl-4-phenyl-piperidine-1 -carboxylic acid tert-butyl ester. Triethylamine (14.0 mL, 100.4 mmol, 3.9 equivalents) was added to a solution of (−)-(3S,4R)-3-hydroxymethyl-4-phenyl-piperidine-1-carboxylic acid tert-butyl ester (7.51 g, 25.8 mmol) in CH₂Cl₂ (185 mL). Methanesulfonyl chloride (3.0 mL, 38.6 mmol, 1.5 equivalents) was slowly added, and the resulting solution was stirred for 24 hours. The reaction was diluted with CH₂CI₂ and 1M HCl. The organic layer was separated, dried (MgSO₄), and rotary evaporated in vacuo to provide a yellow orange solid, 9.97 g (>100%). MS (APCI): MH+=370.1. The crude product was used without purification in the following step.

Intermediate 2: (−)-(3S,4R)-3-(2-Ethoxy-pyridin-3-yloxymethyl)-4-pheny-4-piperidine-1-carboxylic acid tert-butyl ester. Sodium hydride (0.036 g, 1.50 mmol, 1.1 equivalents) was added to a solution of 2-ethoxy-pyridin-3-ol (0.182 g, 1.45 mmol, 1.06 equivalents) in DMF (dimethylformamide) (5 mL). The reaction was stirred for 10 minutes followed by addition of Intermediate 1 (0.503 g, 1.36 mmol). The reaction was sealed and heated to 105° C. for 18 hours. The reaction was cooled to room temperature, quenched with a small amount of water and concentrated in vacuo. The residue was purified by silica gel chromatography with 25% ethyl acetate/hexanes to provide a colorless oil, 0.4513 g (83%). MS (APCI): MH+=413.2.

(−)-(3S,4R)-2-Ethoxy-3-(4-phenyl-piperidin-3-ylmethoxy)-pyridine, free base. Trifluoroacetic acid (1.0 mL, 13.0 mmol, 13 equivalents) was added to a solution of Intermediate 2 (0.447 g, 1.08 mmol) in CH₂Cl₂ (5 mL). The reaction was stirred for 18 hours and then solvent removed in vacuo. The resulting residue was partitioned between ethyl acetate/10% NH₄OH. The aqueous layer was extracted three times with ethyl acetate. The combined organic layers were dried with MgSO₄ and concentrated in vacuo to provide a pale pink oil, 0.3071 g (87%). Optical rotation =−0.1056 (23.4° C., 589 nm). Specific rotation [α]=−84.5 (c=5, CH₃OH). MS (APCI): MH+=313.1.

### Example 1

(−)-(3R,4S)-2-Ethoxy-3-(4-phenyl-piperidin-3-ylmethoxy)-pyridine, fumaric acid salt. To a solution of (−)-(3S,4R)-2-ethoxy-3-(4-phenyl-piperidin-3-ylmethoxy)-pyridine (0.2974 g, 0.953 mmol) in dry acetone (about 3 mL) was added a solution of fumaric acid (0.111 g, 0.956 mmol, 1.0 equivalents) in dry acetone (13 mL). The mixture was stirred overnight. The resulting white precipitate was filtered, rinsed with a small amount of dry acetone and dried to provide the title compound as a white solid, 0.3168 g (82%). MS (APCI): MH+=313.1.

The compounds of Examples 7, 9, 11, 12, and 14-35 were made in a manner analogous to Example 1 by using an appropriately substituted pyridinol, phenol, hydroxy-pyrrazole, hydroxy-pyrazine, or hydroxy-thieno[3,2-b]pyridine in the synthesis of Intermediate 2.

The compounds of Examples 2-6, 8, 10, and 13 were made in a manner analogous to Example 1 by using an appropriately substituted pyridinol, phenol, or hydroxy-pyrrazole, in the synthesis of Intermediate 2, and by using (3R,4S)-3-hydroxymethyl-4-phenyl-piperidine-1-carboxylic acid tert-butyl ester instead of (3S,4R)-3-hydroxymethyl-4-phenyl-piperidine-1-carboxylic acid tert-butyl ester in the synthesis of intermediate 1.

### Example 2

(3R,4S)-2-Ethoxy-3-(4-phenyl-piperidin-3-ylmethoxy)-pyridine, fumarate.

### Example 3

(3R,4S)-3-(2-Ethoxy-phenoxymethyl)-4-phenyl-piperidine, fumarate.

### Example 4

(3R,4S)-2-(4-Phenyl-piperidin-3-ylmethoxy)-5-trifluoromethyl-pyridine, fumarate.

### Example 5

(3R,4S)-5-Methyl-2-(4-phenyl-piperidin-3-ylmethoxy)-pyridine, fumarate.

### Example 6

(3R,4S)-2-Methoxy-3-(4-phenyl-piperidin-3-ylmethoxy)-pyridine, fumarate.

### Example 7

(3S,4R)-2-Methoxy-3-(4-phenyl-piperidin-3-ylmethoxy)-pyridine, fumarate.

### Example 8

(3R,4S)-3-(4-Phenyl-piperidin-3-ylmethoxy)-2-propoxy-pyridine, fumarate.

### Example 9

(3S,4R)-3-(4-Phenyl-piperidin-3-ylmethoxy)-2-propoxy-pyridine, fumarate.

### Example 10

(3R,4S)-2-(2-Methoxy-ethoxy)-3-(4-phenyl-pipe rid in-3-ylmethoxy)-pyridine, fumarate.

### Example 11

(3S,4R)-2-(2-Methoxy-ethoxy)-3-(4-phenyl-pipe rid in-3-ylmethoxy)-pyridine, fumarate.

### Example 12

(3S,4R)-2-lsopropoxy-3-(4-phenyl-piperidin-3-ylmethoxy)-pyridine, fumarate.

### Example 13

(3R,4S)-3-(1-Methyl-5-trifluoromethyl-1 H-pyrazol-3-yloxymethyl)-4-phenyl-piperidine, fumarate.

### Example 14

(3S,4R)-2-(3-Methoxy-propoxy)-3-(4-phenyl-piperidin-3-ylmethoxy)-pyridine, fumarate.

### Example 15

(3S,4R)-2-lsobutoxy-3-(4-phenyl-piperidin-3-ylmethoxy)-pyridine, fumarate.

### Example 16

(3S,4R)-3-(4-Phenyl-piperidin-3-ylmethoxy)-2-(tetrahydro-pyran-4-yloxy)-pyridine, fumarate.

### Example 17

(3S,4R)-3-(4-Phenyl-piperidin-3-ylmethoxy)-2-propyl-pyridine, fumarate.

### Example 18

(3S,4R)-3-(2-Ethoxy-phenoxymethyl)-4-phenyl-piperidine, fumarate.

### Example 19

(3S,4R)-2-(4-Phenyl-piperidin-3-ylmethoxy)-5-trifluoromethyl-pyridine, fumarate.

### Example 20

(3S,4R)-5-Methyl-2-(4-phenyl-piperidin-3-ylmethoxy)-pyridine, fumarate.

### Example 21

(3S,4R)-3-(2-Methyl-5-trifluoromethyl-2H-pyrazol-3-yloxymethyl)-4-phenyl-piperidine, fumarate.

### Example 22

(3S,4R)-2-Methyl-5-(4-phenyl-piperidin-3-ylmethoxy)-pyridine, fumarate.

### Example 23

(3S,4R)-3-(5-Methoxymethyl-2H-pyrazol-3-yloxymethyl)-4-phenyl-piperidine, fumarate.

### Example 24

(3S,4R)-3-(1-Methyl-5-trifluoromethyl-1 H-pyrazol-3-yloxymethyl)-4-phenyl-piperidine, fumarate.

### Example 25

(3S,4R)-3-(4-Phenyl-piperidin-3-ylmethoxy)-benzonitrile, fumarate.

### Example 26

(3S,4R)-4-(4-Phenyl-piperidin-3-ylmethoxy)-benzonitrile, fumarate.

### Example 27

(3S,4R)-3,5-Diisopropyl-4-(4-phenyl-piperidin-3-ylmethoxy)-benzamide, fumarate.

### Example 28

(3S,4R)-2-Methyl-3-(4-phenyl-piperidin-3-ylmethoxy)-pyrazine, fumarate.

### Example 29

(3S,4R)-N-Methyl-4-(4-phenyl-piperid in-3-ylmethoxy)-benzamide, fumarate.

### Example 30

(3S,4R)-3-Methoxy-N-methyl-4-(4-phenyl-piperidin-3-ylmethoxy)-benzamide, fumarate.

### Example 31

(3S,4R)-2-Methoxymethyl-3-(4-phenyl-piperidin-3-ylmethoxy)-pyridine, fumarate.

### Example 32

(3S,4R)-N,N-Dimethyl-4-(4-phenyl-piperidin-3-ylmethoxy)-benzamide, fumarate.

### Example 33

(3S,4R)-3—CHloro-4-(4-phenyl-piperidin-3-ylmethoxy)-benzamide, fumarate.

### Example 34

(3S,4R)-3,5-Dimethyl-4-(4-phenyl-piperidin-3-ylmethoxy)-benzamide, fumarate.

### Example 35

(3S,4R)-7-(4-Phenyl-piperidin-3-ylmethoxy)-thieno[3,2-b]pyridine, free base. Example 35 was made in a manner analogous to Example 1 except the last step that used fumaric acid and acetone was omitted.

Intermediate 3: (−)-(3S,4R)-3-(4-Cyano-2-methoxy-phenoxymethyl)-4-phenyl-piperidine-1-carboxylic acid tert-butyl ester. 4-Hydroxy-3-methoxy-benzonitrile (55 mg, 0.370 mmol, 1.4 equivalents) was treated with TBD-methyl polystyrene basic resin (TBD is 7-methyl-1,5,7- triazabicyclo[4.4.0]dec-5-ene) (1.1 mmol, 440 mg, Novabiochem®, EMD Biosciences, Inc., San Diego, Calif.), suspended in DMF (1.5 mL) and shaken for 2 hours. Intermediate 1 (92 mg, 0.25 mmol, 1 equivalents) in DMF (1 mL) was added and the reaction mixture was heated to 60° C. After shaking for 42 hours, the reaction mixture was cooled, the resin was filtered and washed with acetonitrile. The filtrate was concentrated and the residue to carried on without purification.

### Example 36

(3S,4R) 3-Methoxy-4-(4-phenyl-piperidin-3-ylmethoxy)-benzonitrile, fumaric acid salt. Intermediate 3 was dissolved in dichloromethane (1 mL) and treated with HCl in dioxane (4.0 M, 0.250 mL, 1.0 mmol, 4 equivalents). The reaction mixture was sealed and stirred at ambient temperature for 16 hours. After concentration, the residue was purified by reverse phase HPLC on a Phenomenex, Gemini column (90% water:acetonitrile to 5% water:acetonitrile, spiked with 0.1 % saturated aqueous NH₄OH, to afford 40 mg (50%) of the desired product. The product was dissolved in CH₃OH, treated with fumaric acid (15 mg, 1.05 equivalents), and the mixture was sonicated until homogenous. The clear solution was concentrated to an oil. Evaporation several times from ethyl acetate afforded a white solid which was dried under vacuum to afford 55 mg of the desired product as its fumaric acid salt.

Examples 37-39 were made in a manner analogous to Example 36 by using an appropriately substituted pyridinol, or phenol in the synthesis of Intermediate 3.

### Example 37

(3S,4R)-5—CHloro-2-(4-phenyl-piperidin-3-ylmethoxy)-benzamide, fumarate.

### Example 38

(3S,4R)-4-(4-Phenyl-piperidin-3-ylmethoxy)-benzamide, fumarate.

### Example 39

(3S,4R)-3-(4-Phenyl-piperidin-3-ylmethoxy)-pyridine-2-carboxylic acid amide, fumarate.

Intermediate 4: (−)-(3S,4R)- 3-(2-Methanesulfonyl-phenoxymethyl)-4-phenyl-piperidine-1-carboxylic acid tert-butyl ester. 2-Methanesulfonyl-phenol (64 mg, 0.370 mmol, 1.4 equivalents) was treated with TBD-methyl polystyrene basic resin (1.1 mmol, 440 mg, Novabiochem®, EMD Biosciences, Inc., San Diego, Calif.), suspended in DMF (1.5 mL) and shaken for 16 hours. Intermediate 1 (92 mg, 0.25 mmol, 1 equivalents) in DMF (1 mL) was added and the reaction mixture was heated to 75° C. After shaking for 16 hours, the reaction mixture was cooled, the resin was filtered and washed with DMF. The filtrate was concentrated and the residue was purified by silica gel chromatography (15-40% ethyl acetate:hexanes over 800 mL) to afford 82 mg (74%) of desired product: MS (APCI): M+H =446.1, M-Boc =346.1.

### Example 40

(−)-(3S,4R)-3-(2-Methanesulfonyl-phenoxymethyl)-4-phenyl-piperidine, fumaric acid salt. Intermediate 4 (0.082 g, 0.184 mmol) was dissolved in methanol:dichloromethane (1:1, 2 mL) and treated with HCl in ether (2M, 0.30 mL, 0.60 mmol). The reaction mixture was sealed, stirred at ambient temperature for 16 hours, and concentrated. The residue was partitioned between dichloromethane and saturated aqueous sodium carbonate. The organic layer was separated and the aqueous layer was extracted again with dichloromethane. The combined organic layers were dried over sodium sulfate, filtered and concentrated to an oil. The product was dissolved in CH₃OH, treated with fumaric acid (21 mg), and the mixture was sonicated until homogenous. The clear solution was concentrated to an oil. Evaporation several times from ethyl acetate afforded a white solid which was dried under vacuum to afford 58 mg of the desired product as its fumaric acid salt.

Examples 41-42 were made in a manner analogous to Example 40 by using an appropriately substituted phenol in the synthesis of Intermediate 4.

### Example 41.

(3S,4R)-3-(2-Fluoro-6-methoxy-phenoxymethyl)-4-phenyl-piperidine, fumarate.

### Example 42

(3S, 4R)-2-(4-Phenyl-piperidin-3-ylmethoxy)-benzonitrile, fumarate.

Intermediate 5: (3S,4R)-3-(3-Ethoxy-pyridi n-2-yloxymethyl)-4-phenyl-piperid ine-1 -carboxylic acid tert-butyl ester. Sodium hydride (0.17 g, 4.2 mmol, 1.1 equivalents) was washed twice with hexanes (1 mL). Dimethylformamide (4.5 mL) was added followed by (−)-(3S,4R)-3-hydroxymethyl-4-phenyl-piperidine-1 -carboxylic acid tert-butyl ester (1.1 g, 3.8 mmol). After gas evolution slowed at room temperature, the reaction mixture was heated at 50° C. until gas evolution ceased. To a one-third portion of the resulting solution was added 2—CHloro-3-ethoxypyridine (0.30 g, 1.9 mmol, 1.5 equivalents), and this mixture was heated at 58° C. for 66 hours, then at 100° C. for seven hours. The volatile solvents were removed in vacuo, purging once with ethyl acetate. The residue was partitioned between water and ethyl ether. The ether layer was filtered through sodium sulfate and concentrated in vacuo. The residue was purified by silica gel chromatography (0-60% ethyl acetate:hexanes) to provide a colorless oil, 0.35 g (67%). MS (APCI): MH+=413.2. (3S,4R)-3-Ethoxy-2-(4-phenyl-piperidin-3-ylmethoxy)-pyridine, free base Trifluoroacetic acid (2.4 mL) was added to a ice-cold solution of (3S,4R)-3-(3-ethoxy-pyridin-2-yloxymethyl)-4-phenyl-piperidine-1-carboxylic acid tert-butyl ester (0.35 g, 0.85 mmol) in CH₂Cl₂ (3.6 mL). The reaction was stirred for 45 minutes at ice bath temperature, then two hours at room temperature. The volatile solvents were removed in vacuo, and the residue was partitioned between 10 mL methylene chloride and 1 mL 15% sodium hydroxide aqueous solution. The organic layer was filtered through sodium sulfate and concentrated in vacuo to provide an oil, 0.30 g. MS: MH+=313.1.

### Example 43

(3S,4R)-3-Ethoxy-2-(4-phenyl-piperidin-3-ylmethoxy)-pyridine, fumaric acid salt. To a solution of (3S,4R)-3-ethoxy-2-(4-phenyl-piperidin-3-ylmethoxy)-pyridine (0.3 g, 1 mmol) in dry acetone (about 3 mL) was added a solution of fumaric acid (0.09 g, 0.8 mmol, 0.9 equivalents) in dry acetone (15 mL). The mixture was stirred overnight. The resulting white precipitate was filtered, rinsed with a small amount of dry acetone and dried to provide the title compound as a white solid, 0.145 g (41%).

Examples 44-45 were made in a manner analogous to Example 43 by using an appropriately substituted pyridinol, or hydroxy-pyrazine in the synthesis of Intermediate 5.

### Example 44

(3S,4R)-3-Methoxy-2-(4-phenyl-piperidin-3-ylmethoxy)-pyridine, fumarate.

### Example 45

(3S,4R)-2-Methoxy-3-(4-phenyl-piperidin-3-ylmethoxy)-pyrazine, fumarate.

Intermediate 6: (−)-(3S,4R)- 3-(6-Methyl-pyridin-2-yloxymethyl)-4-phenyl-piperidine-1-carboxylic acid tert-butyl ester. To a solution of 6-methyl-2-pyridinol in acetonitrile was added cesium carbonate then Intermediate 1 and the reaction mixture was stirred at 80° C. overnight. The solvent was concentrated under reduced pressure and the crude solid was taken up in dichloromethane and filtered through a very small pad of silica gel (2 mm). The filtrate was concentrated under reduced pressure giving 302 mg of the product. MS (APCI): MH+=383.3.

### Example 46

(−)-(3S,4R)- 2-Methyl-6-(4-phenyl-piperidin-3-ylmethoxy)-pyridine, fumaric acid salt. The title product of Example 46 was made in a manner analogous to Example 1 by using Intermediate 6 instead of Intermediate 2.

| | MS and/or Combustion analysis (CHN) | |
|---|---|---|
| | (Calculated, Experimental) and/or | |
| | Specific Rotation (free base, | |
| Ex. | 589 nm, CH₃OH, 24° C., c = 5) | ¹HNMR |
| 1 | C₁₉H₂₄N₂O₂ × 0.92C₄H₄O₄ × 0.12C₂H₁F₃O₂ | ¹H NMR(400MHz, DMSO-d₆) [symbol] ppm1.3(t, J=7.0Hz, |
| | Calc: C, 63.59; H, 6.47; N, 6.47. | 3H)1.8(m, 2H)2.4(m, 1H)2.7(td, J=11.7, 4.2Hz, |
| | Found: C, 63.21; H, 6.59; N, 6.17. | 1H)2.8(m, 2H)3.3(m, 1H)3.5(dd, J=12.6, 3.6Hz, |
| | Specific Rotation: −84.5 | 1H)3.6(dd, J=10.0, 7.4Hz, 1H)3.6(m, 1H)4.3 |
| | | (m, 2H)6.5(s, 2H, fumaric acid) 6.8 (dd, J=7.7, |
| | | 5.0Hz, 1H)7.0(dd, J=7.8, 1.4Hz, 1H)7.2(m, 3H)7.3 |
| | | (m, 2H)7.6(dd, J=5.1, 1.6Hz, 1H) |
| 2 | C₁₉H₂₄N₂O₂ × 0.95 C₄H₄O₄ × 0.14 C₂H₁F₃O₂ | ¹H NMR(400MHz, DMSO-d₆)δppm1.3(t, J=7.0Hz, |
| | Calc: C, 63.20; H, 6.42; N, 6.39. | 3H)1.8(m, H)1.8(1.9(m, 1H)2.4(ddd, J=11.3, |
| | Found: C,62.90; H, 6.54; N, 6.18. | 7.3, 3.9Hz, 1H)2.7(td, J=11.7, 4.1Hz, 11H)2.9(m, |
| | Specific Rotation: +85.0 | 2H)2.9(s, 1H)3.3(d, J=12.3Hz, 1H)3.5(dd, |
| | | J=12.4, 4.0Hz, 1H)3.6(dd, J=10.1, 7.3Hz, 1H)3.7 |
| | | (m, 1H)4.3(m, 2H)6.5(s, 2H, fumaric acid)6.8 |
| | | (dd, J=7.7, 5.0Hz, 1H)7.0(dd, J=7.8, 1.6Hz, 1H) |
| | | 7.2(d, J=1.4Hz, 1H)7.2(ddd, J=4.9, 2.4, 2.2Hz, |
| | | 2H)7.3(m, 2H)7.6(dd, J=5.0, 1.5Hz, 1H) |
| 3 | M+ = 312.1 | ¹H NMR(400MHz, DMSO-d₆) [symbol] ppm1.3(m, |
| | | 3H)1.8(t, J=15.3Hz, 1H)1.9(m, 1H)2.4(m, 1H)2.8 |
| | | (m, 1H)2.9(t, J=12.4Hz, 2H)3.3(d, J=12.1Hz, |
| | | 1H)3.5(m, 2H)3.6(m, 1H)4.0(m, 2H)6.5(s, 1H) |
| | | 6.7(m, 2H)6.8(m, 1H)6.9(m, 1H)7.2(m, 2H)7.3 |
| | | (m, 2H) |
| 4 | M+ = 337.1 | ¹H NMR(400MHz, DMSO-d₆) [symbol] ppm 1.8(m, |
| | | 1H)1.9(m, 1H)2.8(m, 1H)2.9(m, 2H)3.3(d, J=14.0Hz, |
| | | 1H)3.5(d, J=16.0Hz, 1H)3.9(m, 1H)4.0(d, |
| | | J=10.9Hz, 1H)6.5(s, 1H)6.9(d, J=8.6Hz, 1H)7.2 |
| | | (d, J=8.2Hz, 3H)7.3(m, 2H)8.0(d, J=8.8Hz, 1H) |
| | | 8.4(s, 1H) |
| 5 | M+ = 283.2 | ¹H NMR(400MHz, DMSO-d₆) [symbol] ppm1.8(d, |
| | | J=12.1Hz, 1H)1.9(m, 1H)2.3(s, 3H)2.4(m, 1H)2.8(t, |
| | | J=12.0Hz, 1H)2.9(m, 2H)3.3(d, J=12.7Hz, 1H) |
| | | 3.4(d, J=16.2Hz, 1H)3.6(dd, J=9.8, 6.5Hz, 1H) |
| | | 3.7(m, 1H)6.5(s, 2H)7.1(m, 2H)7.2(m, 3H)7.3 |
| | | (m, 2H)8.0(s, 1H) |
| 6 | C₁₈H₂₂N₂O₂ × 0.92C₄H₄O₄ × 0.14C₂H₁F₃O₂ | ¹H NMR(400MHz, DMSO-d₆) [symbol] ppm1.8(m, |
| | Calc: C, 62.63; H, 6.18; N, 6.65. | 1H)1.9(dd, J=12.8, 4.0Hz, 1H)2.4(m, 1H)2.8(td, |
| | Found: C, 62.26; H, 6.33; N, 6.39. | J=11.8, 4.0Hz, 1H)2.9(m, 2H)3.3(d, J=12.3Hz, |
| | Specific Rotation: +88.8 | 1H)3.5(dd, J=12.6, 3.8Hz, 1H)3.6(m, 2H)3.8(s, |
| | | 3H)6.5(s, 2H, fumaric acid)6.8(dd, J=7.8, 4.9Hz, |
| | | 1H)7.0(dd, J=7.8, 1.6Hz, 1H)7.2(m, 3H)7.3(m, |
| | | 2H)7.7(dd, J=4.9, 1.6Hz, 1H) |
| 7 | C₁₈H₂₂N₂O₂ × 1.11C₄H₄O₄ × 0.23C₂H₁F₃O₂ | ¹H NMR(400MHz, DMSO-d₆) [symbol] ppm1.7(m, |
| | Calc: C, 60.66; H, 5.93; N, 6.18. | 1H)1.8(m, 1H)2.5(m, 1H)2.7(dt, J=12.2, 3.3, |
| | Found: C, 60.26; H, 6.24; N, 6.35. | 1H)2.9(m, 2H)3.3(m, 1H), 3.5(dd, 12.5, 3.7, |
| | Specific Rotation: −85.3 | 1H)3.6(m, 2H)3.8(s, 3H)6.5(s, 2H, fumaric |
| | | acid)6.8(dd, J=7.7, 5.0Hz, 1H)7.0(dd, J=7.9, |
| | | 1.5Hz, 1H)7.2(d, J=6.6Hz, 2H)7.2(s, 1H)7.3(t, |
| | | J=7.4Hz, 2H)7.7(dd, J=5.1, 1.6Hz, 1H) |
| 8 | C₂₀H₂₆N₂O₂ × 1.04C₄H₄O₄ × 0.05C₂H₁F₃O₂ | ¹H NMR(400MHz, DMSO-d₆) [symbol] ppm0.9(t, |
| | Calc: | J=7.4Hz, 3H)1.7(td, J=14.1, 7.4Hz, 2H)1.8(ddd, |
| | C, 64.34; H, 6.72; N, 6.19. | J=11.1, 2.7, 2.6Hz, 1H)1.9(m, 1H)2.3(s, |
| | Found: C, 64.03; H, 6.81; N, 6.05. | 1H)2.4(ddd, J=11.0, 7.5, 3.2Hz, 1H)2.8(td, |
| | Specific Rotation: +87.5 | J=11.6, 3.9Hz, 1H)2.9(m, 2H)3.3(d, J=12.4Hz, |
| | | 1H)3.5(dd, J=12.3, 3.5Hz, 1H)3.6(dd, J=9.9, |
| | | 7.2Hz, 1H)3.7(m, 1H)4.2(m, 2H)6.5(s, 2H, fumaric |
| | | acid)6.8(dd, J=7.8, 4.9Hz, 1H)7.0(dd, J=7.8, |
| | | 1.5Hz, 1H)7.2(m, 1H)7.3(m, 2H)7.3(m, 2H)7.6(dd, |
| | | J=5.1, 1.5Hz, 1H) |
| 9 | C₂₀H₂₆N₂O₂ × 0.98C₄H₄O₄ × 0.19C₂H₁F₃O₂ | ¹H NMR(400MHz, DMSO-d₆) [symbol] ppm0.9(t, |
| | Calc: C, 63.19; H, 6.57; N, 6.07. | J=7.4Hz, 3H)1.7(q, J=6.7Hz, 2H)1.8(m, 1H)1.9(m, |
| | Found: C, 62.85; H, 6.79; N, 5.89. | 1H)2.5(m, 1H)2.7(dt, J=12.7, 1.9Hz, 1H)2.8(m, |
| | Specific Rotation: −88.0 | 28H)3.3(m, 1H)3.5(dd, J=12.5, 3.5Hz, 1H)3.5(dd, |
| | | J=9.9, 7.1Hz, 1H)3.7(m, 1H)4.2(td, J=6.7, |
| | | 2.8Hz, 1H)6.5(s, 2H, fumaric acid)6.8(dd, J=7.7, |
| | | 5.0Hz, 1H)7.0(dd, J=7.8, 1.4Hz, 1H)7.2(m, |
| | | 3H)7.3(m, 2H)7.6(dd, J=4.9, 1.6Hz, 1H) |
| 10 | C₂₀H₂₆N₂O₃ × 1.00C₄H₄O₄ × 0.22 C₂H₁F₃0₂ | ¹H NMR(400MHz, DMSO-d₆) [symbol] ppm1.8(m, |
| | Calc: C, 60.70; H, 6.30; N, 5.79. | 1H)1.9(m, 1H)2.4(m, 1H)2.8(td, J=11.6, 3.8Hz, |
| | Found: C, 60.38; H, 6.55; N, 5.58. | 1H)2.9(td, J=12.5, 3.5Hz, 2H)3.3(s, 3H)3.3(ddd, |
| | Specific Rotation: +87.7 | J=3.5, 1.6, 1.4Hz, 1H)3.5(dd, J=12.6, 3.2Hz, |
| | | 1H)3.6(dd, J=10.1, 7.2Hz, 1H)3.7(m, 3H)4.4(m, |
| | | 2H)6.5(s, 2H, fumaric acid)6.8(dd, J=7.7, 5.0Hz, |
| | | 1H)7.0(dd, J=7.8, 1.4Hz, 1H)7.2(m, 3H)7.3(m, |
| | | 2H)7.6(dd, J=5.1, 1.4Hz, 1H) |
| 11 | C₂₀H₂₆N₂O₃ × 0.97C₄H₄O₄ × 0.11 C₂H₁F₃O₂ | ¹H NMR(400MHz, DMSO-d₆)δppm1.8(m, |
| | Calc: C, 61.91; H, 6.47; N, 5.99. | 1H)1.9(m, 1H)2.4(ddd, J=11.4, 7.6, 4.5Hz, |
| | Found: C, 61.55; H, 6.62; N, 5.93. | 1H)2.8(td, J=11.7, 3.9Hz, 1H)2.9(td, J=12.6, |
| | Specific Rotation: −85.6 | 3.8Hz, 2H)3.3(s, 3H)3.3(ddd, J=2.0, 1.5, 1.2Hz, |
| | | 1H)3.5(m, 1H)3.6(dd, J=9.9, 7.2Hz, 1H)3.6(t, |
| | | J=4.9Hz, 1H)3.7(m, 2H)4.4(m, 2H)6.5(s, 2H, |
| | | fumaric acid)6.8(dd, J=7.7, 5.0Hz, 1H)7.0(dd, |
| | | J=7.9, 1.5Hz, 1H)7.2(m, 33H)7.3(m, 2H)7.3(s, |
| | | 4H)7.6(dd, J=5.0, 1.5Hz, 1H) |
| 12 | C₂₀H₂₆N₂O₂ × 1.03C₄H₄O₄ × | ¹H NMR(400MHz, DMSO-d₆)δppm1.2(dd, |
| | 0.17 C₂H₁F₃O₂ | J=6.2, 4.5Hz, 6H)1.8(ddd, J=13.9, 6.0, 3.4Hz, |
| | Calc: C, 63.13; H, 6.56; N, 6.02. | 1H)1.9(m, 1H)2.4(m, 1H)2.7(td, J=11.6, 4.1Hz, |
| | Found: C, 62.77; H, 6.81; N, 6.21. | 1H)2.9(m, 2H)3.3(m, 1H)3.5(m, 1H)3.7(dd, J=9.9, |
| | Specific Rotation: −91 .7 | 2.9Hz, 1H)5.2(dt, J=12.3, 6.2Hz, 1H)6.5(s, 2H, |
| | | fumaric acid)6.8(dd, J=7.7, 5.0Hz, 1H)7.0(dd, |
| | | J=7.8, 1.6Hz, 1H)7.2(m, 3H)7.3(m, 2H)7.6(dd, |
| | | J=4.9, 1.6Hz, 1H) |
| 13 | M+ = 340.1 | ¹H NMR(400MHz, DMSO-d₆)δppm1.9(m, |
| | | 2H)2.4(m, 1H)2.8(m, 1H)3.0(m, 2H)3.4(s, 1H)3.5 |
| | | (s, 1H)3.6(s, 3H)3.8(m, 1H)3.8(m, 1H)6.6(s, |
| | | 2H)7.2(m, 3H)7.3(m, 2H)8.7(m, 1H) 8.9(d, |
| | | J=7.2Hz, 1H) |
| 14 | C₂₁H₂₈N₂O₃ × 0.9C₄H₄O₄ × | ¹H NMR(400MHz, DMSO-d₆) [symbol] ppm1.8(dd, |
| | 0.18C₂H₁F₃O₂ | J=13.6, 3.7Hz, 1H)1.9(m, 3H)2.4(m, 1H)2.8(td, |
| | Calc: C, 61.87;H, 6.59; N, 5.71. | J=11.6, 3.9Hz, 1H)2.9(m, 2H)3.2(s, 3H)3.3(m, |
| | Found: C, 61.54; H, 6.72; N, 5.56. | 1H)3.5(t, 2H)3.5(m, 2H)3.7(m, 1H)4.3(m, 2H) |
| | Specific Rotation: −79.2 | 6.5(s, 2H, fumaric acid)6.8(dd, J=7.7, 5.0Hz, 1H) |
| | | 7.0(dd, J=7.8, 1.6Hz, 1H)7.2(m, 3H)7.3(m, 2H) |
| | | 7.6(dd, J=5.1, 1.6Hz, 1H) |
| 15 | C₂₁H₂₈N₂O₂ × 1.02C₄H₄O₄ | ¹H NMR(400MHz, DMSO-d₆)δppm1.8(m, |
| | Calc: C, 65.65; H, 7.05; N, 6.11. | 1H)1.9(m, 2H)2.4(s, 2H)2.4(m, 1H)2.8(td, J=11.6, |
| | Found: C, 65.26; H, 7.13; N, 6.01. | 3.9Hz, 1H)2.9(m, 2H)3.2(m, 1H)3.5(m, 1H)3.5 |
| | Specific Rotation: −132.8 | (m, 1H)3.7(m, 1H)4.3(m, 2H)6.5(s, 2H, fumaric |
| | | acid)6.8(dd, J=7.7, 5.0Hz, 1H)7.0(dd, J=7.8, |
| | | 1.6Hz, 1H)7.2(m, 3H)7.3(m, 2H)7.6(dd, J=5.1, |
| | | 1.6Hz, 1H) |
| 16 | C₂₂H₂₈N₂O₃ × 1.00C₄H₄O₄ × | ¹H NMR(400MHz, DMSO-d₆)δppm1.6(m, |
| | 0.05C₂H₁F₃O₂ | 1H)1.8(m, 1H)1.9(m, 2H)2.4(m, 1H)2.7(dd, J=11.3, |
| | Calc: C, 63.94; H, 6.59; N, 5.71. | 4.1Hz, 1H)2.8(d, J=4.9Hz, 1H)2.8(t, J=12.0Hz, |
| | Found: C, 63.64; H, 6.63; N, 5.74. | 1H)3.3(m, 1H)3.5(m, 4H)3.7(m, 1H)3.8(m, |
| | Specific Rotation: −85.6 | 2H)5.2(dt, J=8.2, 4.1Hz, 1H)6.5(s, 2H, fumaric |
| | | acid)6.8(dd, J=7.8, 4.9Hz, 1H)7.0(dd, J=7.8, |
| | | 1.6Hz, 1H)7.2(s, 1H)7.2(m, 2H)7.3(m, 2H)7.7(dd, |
| | | J=5.0, 1.5Hz, 1H) |
| 17 | C₂₀H₂₆N₂O₁ × 1.00C₄H₄O₄ | ¹H NMR(400MHz, DMSO-d₆)δppm0.9(t, |
| | Calc: C, 67.59; H, 7.09; N, 6.57. | J=7.4Hz, 3H)1.6(dq, J=14.8, 7.5Hz, 2H)1.8(m, |
| | Found: C, 67.40; H, 7.06; N, 6.49. | 1H)1.9(m, 1H)2.3(m, 1H)2.4(s, 4H)2.7(m, 2H)2.8(m, |
| | Specific Rotation: −77.3 | 1H)2.9(m, 2H)3.3(m, 1H)3.5(dd, J=12.8, 3.6Hz, |
| | | 1H)3.6(dd, J=9.7, 5.8Hz, 1H)3.7(m, 1H)6.5(s, 2H, |
| | | fumaric acid)7.0(dd, J=8.4, 1.4Hz, 1H)7.0(m, |
| | | 1H)7.2(m, 3H)7.3(m, 2H)8.0(dd, J=4.7, 1.4Hz, 1H) |
| 18 | MS+ = 312.1 | ¹H NMR(400MHz, DMSO-d₆)δppm1.3(t, |
| | | J=7.0Hz, 3H)1.8(m, 1H)1.9(m, 1H)2.4(m, 1H)2.7(m, |
| | | 1H)2.9(m, 2H)3.3(d, J=11.9Hz, 1H)3.5(m, 2H) |
| | | 3.6(m, 1H)4.0(m, 2H)6.5(s, 2H)6.7(d, J=8.0Hz, |
| | | 1H)6.8(t, J=7.6Hz, 1H)6.8(m, 1H) 6.9(m, 1H) |
| | | 7.2(m, 3H)7.3(m, 2H) |
| 19 | MS+ = 336.35 | ¹H NMR(400MHz, DMSO-d₆)δppm1.9(m, |
| | | 2H)2.8(m, 1H)2.9(m, 2H)3.3(m, 2H)3.5(m, 1H)3.9 |
| | | (m, 1H)4.0(m, 1H)6.5(s, 2H)6.9(d, J=8.8Hz, |
| | | 1H)7.2(m, 3H)8.0(d, 1H)8.4(s, 1H) |
| 20 | MS+ = 283.0 | ¹H NMR(400MHz, DMSO-d₆) [symbol] ppm1.8(m, |
| | | 1H)1.9(m, 1H)2.3(s, 3H)2.4(m, 1H)2.8(m, 1H)2.9(m, |
| | | 2H)3.3(d, J=12.3Hz, 1H)3.5(d, J=9.2Hz, |
| | | 1H)3.6(m, 1H)3.7(m, 1H)6.5(s, 2H)7.1(m, 2H)7.2(m, |
| | | 3H)7.3(t, J=7.3Hz, 2H)8.0(s, 1H) |
| 21 | MS+ = 340.31 | ¹H NMR(400MHz, DMSO-d₆)δppm1.8(m, |
| | | 2H)2.4(m, 1H)2.8(m, 3H)3.3(m, 2H)3.4(m, 1H)3.6 |
| | | (s, 3H)3.8(m, 2H)5.9(s, 1H)6.4(s, 2H)7.2(t, |
| | | J=7.7Hz, 3H)7.3(m, 2H) |
| 22 | C₁₈H₂₂N₂O₁ × 1.00C₄H₄O₄ | ¹H NMR(400MHz, DMSO-d₆)δppm1.8(m, |
| | Calc: C, 66.32; H, 6.58; N, 7.03. | 1H)1.9(m, 1H)2.3(s, 3H)2.4(m, 1H)2.8(td, J=11.7, |
| | Found: C, 64.55; H, 6.64; N, 6.63. | 3.8Hz, 1H)2.9(m, 2H)3.3(m, 1H)3.4(dd, J=12.3, |
| | Specific Rotation: −64.0 | 3.7Hz, 1H)3.6(dd, J=9.9, 6.4Hz, 1H) 3.7(m, 1H) |
| | | 6.5(s, 2H, fumaric acid)7.0(m, 2H)7.2(m, |
| | | 3H)7.3(m, 2H)8.0(dd, J=2.5, 1.0Hz, 1H) |
| 23 | MS+ = 302.1 | ¹H NMR(400MHz, DMSO-d₆)δppm1.6(m, |
| | Specific Rotation: −56.8 | 2H)2.1(m, 1H)2.4(m, 1H)2.6(m, 1H)2.9(m, |
| | | 1H)3.0(s, 3H)3.1(m, 1H)3.2(s, 1H)3.6(m, |
| | | 1H)3.7(m, 1H) 4.2(s, 2H)5.5(s, 1H)7.2(m, |
| | | 3H)7.3(m, 2H) |
| 24 | MS+ = 340.1 | ¹H NMR(400MHz, DMSO-d₆)δppm1.8(s, |
| | Specific Rotation: −37.6 | 2H)2.4(s, 1H)2.8(m, 4H)3.2(m, 2H)3.4(m, |
| | | 1H)3.7(m, 3H)3.7(s, 1H)6.2(s, 1H)7.2(m, |
| | | 3H)7.3(m, 2H) |
| 25 | CHN: Calculated for C₁₉H₂₀N₂O, | ¹H NMR(400MHz, METHANOL-d₄)δ |
| | 1.05 eq fumaric acid = 67.27%C, | ppm2.02-2.10(m, 2H)2.41-2.53(m, |
| | 5.89%H, 6.76%N. | 1H)2.91-3.01(m, 1H)3.10-3.21(m, 2H)3.52(br d, |
| | Found = 66.93%C, 5.77%H, 6.71%N. | J=12.87Hz, 1H)3.65-3.73(m, 2H)3.76-3.82(m, |
| | | 1H)6.68(s, 2H)7.06-7.12(m, 2H)7.22-7.29(m, |
| | | 4H)7.29-7.43(m, 3H) |
| 26 | CHN: Calculated for C₁₉H₂₀N₂O, | ¹H NMR(400MHz, METHANOL-d₄)δ |
| | 1.05 eq fumaric acid = 67.27%C, | ppm2.02-2.10(m, 2H)2.43-2.55(m, |
| | 5.89%H, 6.76%N. | 1H)2.90-3.01(m, 1H)3.11-3.21(m, 2H)3.52(br d, |
| | Found = 67.31%C, 5.91%H, 6.73%N. | J=12.28Hz, 1H)3.63-3.76(m, 2H)3.78-3.84(m, |
| | | 1H)6.69(s, 2H)6.91(d, J=9.16Hz, 1H)7.20-7.36(m, |
| | | 5H)7.59(d, J=8.19Hz, 1H) |
| 27 | CHN: Calculated for C₁₉H₂₀N₂O, | ¹H NMR(400 MHz, METHANOL-d₄)δ |
| | 0.90 eq fumaric acid = 68.84%C, | ppm1.07(t, J=6.73Hz, 12H)1.89-2.10(m, |
| | 7.59%H, 5.61%N. | 2H)2.85-3.02(m, 3H)3.21-3.28(m, 1H)3.41(dd, |
| | Found = 68.69%C, 7.89%H, 5.77%N. | J=9.45, 2.83Hz, 1H)3.48(d, J=12.87Hz, |
| | | 1H)3.61(dd, J=9.75, 7.02Hz, 1H)3.86(dd, |
| | | J=12.09, 3.51Hz, 1H)6.68(s, 1H)7.23-7.40(m, |
| | | 5H)7.57(s, 2H) |
| 28 | MS (APCI): M + H = 284.2 | ¹H NMR(400MHz, METHANOL-d₄)δ |
| | | ppm1.98-2.10(m, 2H)2.42(s, 3H)2.51-2.61(m, |
| | | 1H)2.91(ddd, J=11.40, 4.48Hz, 1H)3.07-3.20(m, |
| | | 2H)3.46-3.54(m, 1H)3.65-3.72(m, 1H)3.99(dd, |
| | | J=11.31, 7.02Hz, 1H)4.19(dd, J=11.31, 3.12Hz, |
| | | 1H)6.68(s, 2H)7.22-7.35(m, 5H)7.84-7.89(m, |
| | | 1H)7.93(d, J=2.73Hz, 1H) |
| 29 | MS (APCI): M + H = 325.2 | ¹H NMR(400 MHz, METHANOL-d₄)δ |
| | | ppm1.98-2.06(m, 2H)2.37-2.48(m, 1H)2.86(s, |
| | | 3H)2.87-2.96(m, 1H)3.04-3.14(m, |
| | | 2H)3.42-3.50(m, 1H)3.60-3.66(m, 1H)3.68(dd, |
| | | J=9.84, 6.34Hz, 1H)3.75-3.80(m, 1H)6.68(s, |
| | | 1H)6.80(d, J=8.97Hz, 2H)7.19-7.35(m, |
| | | 5H)7.69(d, J=8.77Hz, 2H) |
| 30 | MS (APCI): M + H = 355.3 | ¹H NMR(400MHz, METHANOL-d₄)δ |
| | | ppm1.99-2.08(m, 2H)2.87(s, 3H)2.91-3.01(m, |
| | | 1H)3.08-3.22(m, 2H)3.45-3.53(m, |
| | | 1H)3.64-3.73(m, 2H)3.80(dd, J=9.84, 2.83Hz, |
| | | 1H)3.87(s, 3H)6.67(s, 2H)6.70(s, 0.5H)7.20-7.34(m, |
| | | 6H) |
| 31 | C₁₉H₂₄N₂O₂ × 1.07C₄H₄O₄ | ¹H NMR(400MHz, DMSO-d₆)δppm1.86(1H, d, |
| | Calc: C, 64.04; H, 6.53; N, 6.42. | J=11.9Hz), 1.99(1H, m), 2.86(1H, m), 2.93(3H, |
| | Found: C, 64.04; H, 6.34; N, 6.36. | td, J=12.5, 4.7Hz), 3.34(3H, m), 3.51(1H, m), 3.58 |
| | | (1H, dd, J=9.8, 5.6Hz), 3.75(1H, dd, J=9.7, |
| | | 2.3Hz), 4.49(2H, m), 6.48(2H, s), 7.12(1H, d, J=0.8Hz), |
| | | 7.20(1H, d, J=4.7Hz), 7.22(4H, d, J=7.4Hz), |
| | | 7.29(2H, d, J=7.2Hz), 7.32(1H, s), 8.06(1H, dd, |
| | | J=4.7, 1.0Hz) |
| 32 | MS (APCI): M + H = 339.2 | ¹H NMR(400MHz, METHANOL-d₄)δ |
| | | ppm2.02-2.10(m, 2H)2.42-2.54(m, 1H)2.91- |
| | | 3.09(m, 7H)3.11-3.22(m, 2H)3.53(d, J=12.28Hz, |
| | | 1H)3.65-3.73(m, 2H)3.76-3.81(m, 1H)6.69(s, |
| | | 2H)6.83(d, J=8.97Hz, 2H)7.20-7.37(m, 7H) |
| 33 | MS (APCI): M + H = 345.2 | ¹H NMR(400MHz, METHANOL-d₄)δ |
| | | ppm2.03-2.12(m, 2H)2.46-2.57(m, 1H)2.97- |
| | | 3.06(m, 1H)3.09-3.16(m, 1H)3.20(t, J=12.38Hz, |
| | | 1H)3.53(d, J=13.26Hz, 1H)3.68-3.78(m, |
| | | 2H)3.91(dd, J=9.84, 2.63Hz, 1H)6.68(s, |
| | | 1.2H)6.81(d, J=8.58Hz, 1H)7.21-7.35(m, |
| | | 5H)7.70(dd, J=8.58, 2.34Hz, 1H)7.92(d, |
| | | J=2.14Hz, 1H) |
| 34 | MS (APCI): M + H = 339.2 | ¹H NMR(400MHz, METHANOL-d₄)δ |
| | | ppm1.87-2.03(m, 2H)2.06(s, 6H)2.48-2.59(m, |
| | | 1H)2.82(ddd, J=11.78, 4.03Hz, 1H)3.07(ddd, |
| | | J=12.82, 3.66Hz, 1H)3.17(t, J=12.09Hz, |
| | | 1H)3.37-3.47(m, 2H)3.60(dd, J=9.53, 7.57Hz, |
| | | 1H)3.84(dd, J=12.70, 3.18Hz, 1H)6.67(s, |
| | | 1H)7.21-7.36(m, 5H)7.44(s, 2H) |
| 35 | MS (APCI) MH+ = 325.1 | ¹H NMR(400MHz, DMSO-d₆)δppm1.7(m, |
| | | 2H)2.2(m, 1H)2.6(s, 6H)2.6(m, 2H)3.1(dt, J=12.2, |
| | | 3.0Hz, 1H)3.3(dd, J=12.0, 3.8Hz, 2H)3.8(dd, |
| | | J=9.9, 7.0Hz, 1H)3.9(m, 1H)6.6(d, J=5.5Hz, 1H) |
| | | 7.2(m, 1H)7.3(m, 4H)7.5(d, J=5.5Hz, 1H)8.0(d, |
| | | J=5.5Hz, 1H)8.4(d, J=5.5Hz, 1H) |
| 36 | MS (APCI): M + H = 323.1 | ¹H NMR(400MHz, METHANOL-d₄)δppm2.1(m, |
| | | 2H)2.5(m, 1H)3.0(dt, J=11.2, 8.3Hz, 1H)3.2(m, |
| | | 2H)3.5(d, J=12.3Hz, 1H)3.7(m, 2H)3.8(dd, J=9.9, |
| | | 2.7Hz, 1H)3.9(s, 3H)6.7(s, 2H)6.8(d, J=8.4Hz, |
| | | 1H)7.2(dd, J=8.4, 1.8Hz, 1H)7.2(m, 4H)7.3(m, |
| | | 2H) |
| 37 | MS (APCI): M + H = 345.1 | ¹H NMR(400MHz, METHANOL-d₄)δppm2.0(m, |
| | | 2H)2.5(m, 1H)2.9(m, 1H)3.1(m, 2H)3.5(d, J=12.9Hz, |
| | | 1H)3.7(dd, J=12.2, 3.6Hz, 1H)3.8(dd, J=9.8, |
| | | 6.3Hz, 1H)3.9(dd, J=9.8, 3.0Hz, 1H)6.7(s, |
| | | 2H)6.8(d, J=9.0Hz, 1H)7.3(m, 3H)7.3(m, 3H) |
| | | 7.6(d, J=2.7Hz, 1H) |
| 38 | CHN: Calculated for C₁₉H₂₂N₂O₂, | ¹H NMR (400 MHz, METHANOL-d₄)δppm2.1(m, |
| | 1.2 eq fumaric acid, 0.15 eq water = | 2H)2.5(m, J=8.4, 6.2Hz, 1H)3.0(m, 1H)3.2(m, |
| | 63.19% C, 6.04%H, 6.19%N. | 2H)3.5(d, J=13.1Hz, 1H)3.7(m, 2H)3.8(m, 1H) |
| | Found = 63.16%C, 6.30%H, 6.12%N. | 6.7(s, 2H)6.8(d, J=8.6Hz, 2H)7.2(m, 3H)7.3(m, |
| | | 2H)7.8(d, J=8.4Hz, 2H) |
| 39 | C₁₈H₂₁N₃O₂ × 1.22C₄H₄0₄ | ¹H NMR(400MHz, DMSO-d₆)δppm1.84(1H, |
| | Calc: C, 60.42; H, 5.73; N, 9.16. | s), 1.95(2H, m), 2.08(1H, s), 2.40(1H, s), 2.85(3H, |
| | Found: C, 60.36; H, 5.63; N, 9.19. | m), 3.32(1H, d, J=12.7Hz), 3.51(1H, dd, J=12.8, |
| | | 3.8Hz), 3.58(1H, dd, J=9.8, 6.3Hz), 3.76(1H, dd, |
| | | J=9.7, 2.5Hz), 6.46(2H, s), 7.21(5H, m), 7.32(3H, |
| | | m), 7.43(1H, s), 7.78(1H, s), 8.09(1H, dd, J=4.6, |
| | | 1.1Hz) |
| 40 | MS (APCI): M + H = 346.1 | ¹H NMR(400MHz, METHANOL-d₄)δppm2.1(m, |
| | | 2H)2.5(m, 1H)3.0(m, 1H)3.1(m, 1H)3.2(m, 4H)3.5(m, |
| | | 1H)3.8(m, 1H)3.9(dd, J=9.8, 6.1Hz, 1H)4.0(m, |
| | | 1H)6.7(s, 2H)6.9(dd, J=8.4, 0.6Hz, 1H)7.1(m, |
| | | 1H)7.3(m, 5H)7.6(ddd, J=8.8, 7.1, 1.7Hz, |
| | | 1H)7.9(dd, J=8.0, 1.8Hz, 1H) |
| 41 | CHN: Calculated for C₁₉H₂₂N₂O₂, | ¹H NMR(400MHz, METHANOL-d₄)δppm2.0(m, |
| | 1.2 eq fumaric acid = 62.87%C, | 2H)2.4(m, 1H)2.9(td, J=11.6, 4.6Hz, 1H)3.1(td, |
| | 5.94%H, 3.08%N. | J=12.4, 4.2Hz, 1H)3.2(d, J=12.3Hz, 1H)3.5(m, |
| | Found = 62.82%C, 6.22%H, 3.15%N. | 1H)3.7(dd, J=9.8, 6.3Hz, 1H)3.7(m, 3H)3.8(m, |
| | | 1H)3.8(dd, J=12.5, 3.3Hz, 1H)6.7(m, 3H)6.8(d, |
| | | J=7.8Hz, 1H)7.0(m, J=8.5, 8.5, 6.1, 0.7Hz, |
| | | 1H)7.2(m, 3H)7.3(m, 2H) |
| 42 | CHN: Calculated for C₁₉H₂₀N₂O, | ¹H NMR(400MHz, METHANOL-d₄)δ |
| | 1.05 eq fumaric acid = 66.22%C, | ppm2.03-2.12(m, 2H)2.47-2.60(m, |
| | 5.79%H, 6.49%N. | 1H)2.95-3.07(m, 1H)3.10-3.27(m, 2H)3.55(br d, |
| | Found = 66.24%C, 5.83% H, 6.45%N. | J=12.67Hz, 1H)3.69-3.81(m, 2H)3.93(dd, |
| | | J=9.75, 2.53Hz, 1H)6.69(s, 2H)6.85(d, J=8.58Hz, |
| | | 1H)7.05(t, J=7.60Hz, 1H)7.20-7.38(m, |
| | | 5H)7.46-7.55(m, 1H)7.61(dd, J=7.70, 1.66Hz, 1H) |
| 43 | MS(APCI): MH+ = 313.1. | ¹H NMR(400MHz, DMSO-d₆)δppm1.3(t, 3H) |
| | C₁₉H₂₄N₂O₂ ×1.00C₄H₄O₄ | 1.8(m, 2H)2.4(m, 1H)2.7(m, 3H)3.4(m, 3H)3.8 |
| | Calc: C, 64.47; H, 6.59; N, 6.54. | (m, 1H)3.9(m, 1H)4.0(q, 2H)6.4(s, 2H, fumaric |
| | Found: C, 64.32; H, 6.22; N, 6.34. | acid)6.8(m, 1H)7.2(m, 4H)7.3(m, 2H)7.5(m, 1H) |
| 44 | MS (APCI) MH+ = 299.2 | ¹H NMR(400MHz, DMSO-d₆)δppm1.8(m, |
| | | 2H)2.5(m, 1H)2.7(m, 4H)3.4(m, 2H)3.8(s, 3H)3.8 |
| | | (m, 1H)3.9(m, 1H)6.4(s, 2H, fumaric acid)6.9(m, |
| | | 1H)7.2(m, 4H)7.3(m, 2H)7.5(m, 1H) |
| 45 | MS (APCI) MH+ = 300.2 | ¹H NMR(400MHz, DMSO-d₆)δppm1.8(m, |
| | | 2H)2.4(m, 1H)2.7(m, 4H)3.4(m, 2H)3.8(m, 1H)3.8(s, |
| | | 3H)3.9(m, 1H)6.4(s, 2H, fumaric acid)7.2(m, |
| | | 3H)7.5(m, 1H)7.6(m, 1H) |
| 46 | C₁₈H₂₂N₂O₁ × 1.02C₄H₄O₄ | ¹H NMR(400MHz, DMSO-d₆)δppm1.83(1H, m), |
| | Calc: C, 66.17; H, 6.56; N, 6.95. | 1.93(1H, dd, J=13.1, 3.3Hz), 2.27(3H, s), 2.55(1H, |
| | Found: C, 66.21; H, 6.67; N, 6.95. | m), 2.76(1H, td, J=11.6, 3.9Hz), 2.91(2H, m), |
| | | 3.30(1H, d, J=12.3Hz), 3.47(1H, dd, J=12.5, |
| | | 3.5Hz), 3.79(1H, dd, J=10.9, 7.6Hz), 4.00(1H, dd, |
| | | J=10.9, 2.9Hz), 6.46(2H, m), 6.54(1H, d, |
| | | J=8.2Hz), 6.77(1H, d, J=7.2Hz), 7.24(3H, m), 7.33(2H, |
| | | t, J=7.4Hz), 7.54(1H, m) |

Intermediate 7: trans-3-(Biphenyl-2-yloxymethyl)-1-methyl-4-phenyl-piperidine. Diisopropyldicarboxylate (250 μL, 1.25 mmol) was added drop wise via syringe to a solution of biphenyl-2-ol (170 mg, 1.0 mmol), trans-(1-methyl-4-phenyl-piperidin-3-yl)-methanol (see Plati et al. (1957) J. Org. Chem. 22: 261-265) (205 mg, 1.0 mmol) and triphenylphosphine (330 mg, 1.25 mmol) in anhydrous THF (4 mL) under nitrogen. The reaction mixture was sealed, stirred at ambient temperature for 16 hours, and concentrated. The residue was purified by preparative scale HPLC to yield 173 mg (52%). MS (APCI): M+H =358.1. ¹ H NMR (400 MHz, CHLOROFORM-d) δ ppm 1.62 (1 H, s), 1.78 (1 H, m), 1.89 (3 H, m), 2.24 (1 H, m), 2.28 (3 H, s), 2.32 (1 H, m), 2.92 (1 H, d, J=9.8Hz), 3.05 (1H, dd, J=11.3, 2.1 Hz), 3.50 (1H, dd, J=9.0, 6.8 Hz), 3.70 (1H, dd, J=9.2, 2.8 Hz), 6.70 (1H, d, J=7.8 Hz), 6.98 (1H, td, J=7.4, 1.0 Hz), 7.09 (2H, d, J=7.1 Hz), 7.20 (1H, m), 7.33 (2H, m), 7.43 (2H, t, J=7.6 Hz), 7.54 (2H, m).

### Example 47

trans-3-(Biphenyl-2-yloxymethyl)-4-phenyl-piperidine hydrochloride salt. An 8 dram screw top vial equipped with stir bar was charged with trans-3-(biphenyl-2-yloxymethyl)-1-methyl-4-phenyl-piperidine (150 mg, 0.42 mmol), methylene chloride (2 mL) and proton sponge (36 mg, 0.17 mmol) followed by α—CHloroethylchloroformate (ACE-Cl) (170 μL, 1.55 mmol). The reaction was warmed to 50° C. for 3 hours then stirred at ambient temperature for 16 hours. Anhydrous hydrogen chloride (5 mL of a 1 N solution in diethyl ether) was added and a precipitate formed over a 15 minute period. The reaction was stirred an additional 30 minutes then filtered through a 2 cm pad of silica gel to remove the precipitate. The resulting solution was concentrated and the oily residue taken up in methanol and stirred at 50° C. for 16 hours. The solvents were removed under reduced pressure and resulting solids triturated with diethyl ether to yield 111 mg (70%) as a white solid. MS (APCI): MH+=344.2. Microanalysis: C₂₄H₂₅NO-1.00HCl-0.30H₂O. Calc: C, 74.81; H, 6.96; N, 3.64. Found: C, 74.60; H, 7.11; N, 3.55.

Examples 48-56 were made in a manner analogous to Example 47 by using an appropriately substituted phenol to replace biphenyl-2-ol in the synthesis of Intermediate 7.

### Example 48

trans-3-(2-Ethoxy-phenoxymethyl)-4-phenyl-piperidine hydrochloride salt. MS (APCI): MH+=312.2. Microanalysis: C₂₀H₂₅NO₂-1.00HCl-0.20H₂O. Calc: C, 68.34; H, 7.57; N, 3.99. Found: C, 68.24; H, 7.65; N, 3.93.

### Example 49

trans-3-(2-lsopropyl-phenoxymethyl)-4-phenyl-piperidine oxalate. MS (APCI): MH+=310.2. Microanalysis: C₂₁ H₂₇NO-1.10C₂H₂O₄. Calc: C, 68.22; H, 7.21; N, 3.43. Found: C, 68.11; H, 7.32; N, 3.39.

### Example 50

trans-4-Phenyl-3-(2-propyl-phenoxymethyl)-piperidine hydrochloride salt. MS (APCI): MH+=310.2. Microanalysis: C₂₁ H₂₇NO-1.00HCl-0.30H₂O. Calc: C, 71.80; H, 8.21; N, 3.99. Found: C, 71.66; H, 8.07; N, 4.07.

### Example 51

trans-3-(2-Cyclohexyl-phenoxymethyl)-4-phenyl-piperidine hydrochloride salt. MS (APCI): MH+=350.3. Microanalysis: C₂₄H₃₁NO-1.00HCl-1.00H₂O. Calc: C, 71.35; H, 8.48; N, 3.47. Found: C, 71.19; H, 8.19; N, 3.93.

Examples 52-56 were made in a manner analogous to Example 47 by using cis-(1-methyl-4-phenyl-piperidin-3-yl)-methanol (see Plati et al. (1957) J. Org. Chem. 22: 261-265) instead of trans-(1-methyl-4-phenyl-piperidin-3-yl)-methanol, and by using an appropriately substituted phenol instead of biphenyl-2-ol in the synthesis of Intermediate 7.

### Example 52

cis-3-(2-Ethoxy-phenoxymethyl)-4-phenyl-piperidine hydrochloride salt. MS (APCI): MH+=312.2. Microanalysis: C₂₀H₂₅NO₂-1.00HCl0.10H₂O. Calc: C, 68.70; H, 7.55; N, 4.01. Found: C, 68.57; H, 7.62; N, 4.12.

### Example 53

cis-4-Phenyl-3-(2-propyl-phenoxymethyl)-piperidine hydrochloride salt. MS (APCI): MH+=310.2. Microanalysis: C₂₁ H₂₇NO-1.00HCl-0.20H₂O. Calc: C, 72.17; H, 8.19; N, 4.01. Found: C, 71.89; H, 8.07; N, 4.07.

### Example 54

cis-3-(2-lsopropyl-phenoxymethyl)-4-phenyl-piperidine hydrochloride salt. MS (APCI): MH+=310.2. Microanalysis: C₂₁ H₂₇NO-1.50HCl. Calc: C, 69.27; H, 7.89; N, 3.85. Found: C, 69.48; H, 7.80; N, 3.81.

### Example 55

cis-3-(Biphenyl-2-yloxymethyl)-4-phenyl-piperidine hydrochloride salt. MS (APCI): MH+=344.2. Microanalysis: C₂₄H₂₅NO-1.00HCl0.10H₂O. Calc: C, 75.51; H, 6.92; N, 3.67. Found: C, 75.12; H, 7.00; N, 3.77.

### Example 56

cis-3-(2-Cyclohexyl-phenoxymethyl)-4-phenyl-piperidine hydrochloride salt. MS (APCI): MH+=350.2. Microanalysis: C₂₄H₃₁ NO1.00HCl-0.20H₂O. Calc: C, 73.99; H, 8.38; N, 3.60. Found: C, 73.83; H, 8.54; N, 3.58.

Intermediate 8. (±)-cis-4-(4-Fluoro-phenyl)-3-hydroxymethyl-piperidine-1-carboxylic acid tert-butyl ester. (±)-cis-4-(4-Fluoro-phenyl)-piperidine-1,3-dicarboxylic acid 1-tert-butyl ester 3-ethyl ester (1 g, 2.85 mmol) was taken up in tetrahydrofuran (5 mL) and added dropwise to a suspension of lithium aluminum hydride (0.12 g, 3.1 mmol) in tetrahydrofuran cooled to 0° C. The reaction mixture was stirred for 2 hours at 0° C., then cautiously quenched with 1 N HCl until a viscous paste was formed and degassing no longer occurred. The reaction mixture was diluted with 25 mL of ethyl acetate and the mixture was filtered through a pad of diatomaceous earth. The filtrate was dried over anhydrous sodium sulfate, filtered, and the filtrate was concentrated to dryness. The crude product was taken up in dichloromethane and purified using silica gel chromatography (elution with 70% hexane/30% ethyl acetate) to give Intermediate 8 as a white solid. Yield: 0.86 g, (86%).¹ H NMR (CDCl₃) δ 1.4 (s, 9H), 1.6 (d, 1 H), 1.9 (m, 1 H), 2.1 (bs,1 H), 2.9 (bs, 2H), 3.1 (m, 2H), 3.4 (bs 1 H), 4.2 (bs,1 H), 4.4 (d,1 H), 7.0 (m, 2H), 7.1 (m, 2H) ppm. Intermediate 9. (±)-cis-4-(4-Fluoro-phenyl)-3-methanesulfonyloxymethyl-piperidine-1-carboxylic acid tert-butyl ester. Intermediate 8 (0.60 g, 1.93 mmol) was taken up in dichloromethane (5 mL) followed by the addition of triethylamine (0.24 g, 2.3 mmol) and methanesulfonyl chloride (0.27 g, 2.3 mmol) via dropwise addition at ambient temperature. The reaction mixture was stirred at room temperature for 4 hours, then diluted with brine and an additional 10 mL of dichloromethane. The organic phase was separated, dried over anhydrous sodium sulfate, filtered and the filtrate was concentrated in vacuo to give Intermediate 9 as a colorless liquid. Trituration with petroleum ether yielded a white solid (0.65 g, 83.8%). ¹ H NMR (CDCl₃) δ 1.5 (s, 9H), 1.7 (m, 1 H), 1.9 (m, 1 H), 2.1 (m, 1 H), 2.6 (m, 1 H), 2.8 (m, 2H), 2.9 (s, 3H), 3.8 (m, 1 H), 4.0 (m, 1 H), 4.2 (bs, 1 H), 4.4 (bs,1 H), 7.0 (m, 2H), 7.1 (m, 2H) ppm.

### Example 57

(+)-cis-2-Ethoxy-3-[4-(4-fluoro-phenyl)-piperidin-3-ylmethoxy]-pyridine fumaric acid salt. To a solution of 2-ethoxy-pyridin-3-ol (0.12 g, 0.85 mmol) in dimethylformamide (5 mL) stirred at room temperature was added sodium hydride (60% by weight, 0.034 g, 0.85 mmol) in several portions. The mixture was stirred for 10 minutes followed by the addition of Intermediate 9 (0.29 g, 0.77 mmol) in a single portion. The solution was refluxed for overnight, cooled to room temperature, and diluted with 15 mL each of water and ethyl acetate. The organic phase was separated, dried over anhydrous sodium sulfate, filtered and the filtrate was concentrated in vacuo to yield cis-3-(2-ethoxy-pyridin-3-yloxymethyl)-4-(4-fluoro-phenyl)-piperidine-1-carboxylic acid tert-butyl ester as a viscous liquid (0.04 g, 12%) using silica gel chromatography (elution with 70% hexane/30% ethyl acetate).

cis-3-(2-Ethoxy-pyrid i n-3-yloxymethyl)-4-(4-fluoro-phenyl)-piperidine-1-carboxylic acid tert-butyl ester (0.04 g, 0.09 mmol) was taken up in dichloromethane (10 mL) followed by the dropwise addition of trifluoroacetic acid (3 mL) at room temperature. The solution was stirred at room temperature for overnight, then concentrated in vacuo. The product was partitioned between saturated sodium bicarbonate and ethyl acetate (5 mL each). The organic phase was separated, dried over anhydrous sodium sulfate, filtered and concentrated to dryness. The resulting viscous liquid was taken up in 3 mL of anhydrous acetone and added dropwise to a solution of fumaric acid in acetone (0.010 g, 0.09 mmol in 5 mL acetone). The solution was stirred at room temperature for overnight affording a white precipitate. The title compound was collected by filtration and dried in vacuo. Yield: 0.027 g, 67%; ¹ H NMR (DMSO-d₆) δ1.3 (t, 3H), 1.7 (d, 1 H), 2.1 (m, 1 H), 2.4 (bs, 1 H), 2.9 (t, 1 H), 3.1 (d, 1 H), 3.2 (m, 1 H), 3.3 (d, 1 H), 3.4 (d, 1 H), 3.5 (dd, 1 H), 4.1 (t, 1 H), 4.3 (q, 2H), 6.4 (s, 2H), 6.8 (m, 1 H), 7.0 (d, 1 H), 7.1 (m, 2H), 7.3 (m, 2H), 7.6 (d, 1 H) ppm. Microanalysis: C₁₉H₂₃FN₂O₂ 1.0 C₄H₄O₄ 0.8 H₂O: %C_{calcd.}59.93 % C_{found}60.00; % H_{calcd.}6.27 % H_{found}6.20; % N_{calcd.}6.08 % N_{found}5.88.

Intermediate 10. (±)-trans- 4-(4-Fluoro-phenyl)-piperidine-1,3-dicarboxylic acid-1 -tert-butyl ester. The compound (±)-cis-4-(4-fluoro-phenyl)-piperidine-1,3-dicarboxylic acid 1-tert-butyl ester 3-ethyl ester, 6 mmol) was suspended in absolute ethanol (25 mL) followed by the addition of solid sodium ethoxide (0.77 g, 11.3 mmol). The reaction mixture was refluxed for 16 hours, cooled to room temperature and concentrated in vacuo. Monitoring the reaction progress by HPLC showed the conversion of the cis to the trans isomer and also conversion of the trans ester to the corresponding carboxylic acid. The crude product was partitioned between dichloromethane (25 mL) and 1N HCl (25 mL), the layers separated, and the organic portion was dried over anhydrous magnesium sulfate and filtered. The filtrate was concentrated to dryness and the crude product was purified using silica gel chromatography (elution with 90% dichloromethane/10% tetrahydrofuran) to give Intermediate 10 (1.2 g, 65%) as a white foam. ¹ H NMR (CDCl₃) δ 1.5 (s, 9H), 1.6 (m, 1 H), 1.8, (d, 1 H), 2.7 (m, 1 H), 2.9 (m, 3H), 4.2 (bs, 1 H), 4.4 (bs, 1 H), 7.0 (m, 2H), 7.1 (m, 2H), ppm.

Intermediate 11. (±)-trans-4-(4-Fluoro-phenyl)-3-hyd roxymethyl-piperidine-1 -carboxylic acid tert-butyl ester. Intermediate 10 (1.2 g, 3.7 mmol) was taken up in tetrahydrofuran (25 mL) followed by the dropwise addition of 1.8M borane in tetrahydrofuran (4.1 mL, 7.4 mmol). The solution was stirred at room temperature and under an atmosphere of nitrogen for 5 days, at which time the reaction mixture was diluted with 1 N sodium hydroxide (20 mL) and dichloromethane (25 mL). The organic phase was separated and dried over anhydrous sodium sulfate. The solution was filtered and the filtrate concentrated to dryness leaving a colorless liquid. The crude product was purified using silica gel chromatography (elution with 70% hexane/30% ethyl acetate). A colorless viscous liquid was obtained which formed a white solid on standing. Yield: 0.91 g, 79% ; ¹ H NMR (CDCl₃) δ 1.5 (s, 9H), 1.6 (m, 1 H), 1.8 (m, 1 H), 2.5 (m, 1 H), 2.6-2.9 (m, 2H), 3.2 (m, 1 H), 3.5 (m, 1 H), 4.2 (d, 1 H), 4.4 (d, 1 H), 7.0 (m, 2H), 7.1 (m, 2H) ppm.

Intermediate 12. (±)-trans-4-(4-Fluoro-phenyl)-3-methanesulfonyloxymethyl-piperidine-1-carboxylic acid tert-butyl ester (4). Intermediate 11 (0.71 g, 2.3 mmol) was taken up in dichloromethane (5 mL) followed by the addition of triethylamine (0.28 g, 2.8 mmol) and methanesulfonyl chloride (0.32 g, 2.8 mmol) via dropwise addition at ambient temperature. The reaction mixture was stirred at room temperature for 4 hours, then diluted with brine and an additional 10 mL of dichloromethane. The organic phase was separated, dried over anhydrous sodium sulfate, filtered and the filtrate was concentrated in vacuo to give the product Intermediate 12 as a colorless liquid. Trituration with petroleum ether yielded a white solid (0.79 g, 87.8%). 1 H NMR (CDCl₃) δ 1.5 (s, 9H), 1.7 (m, 1 H), 1.8 (d, 1 H), 2.0 (m, 1 H), 2.6 (m, 1 H), 2.7 (m, 2H), 2.9 (s, 3H), 3.8 (m, 1 H), 3.9 (m, 1 H), 4.2 (bs, 1 H), 4.4 (bs, 1 H), 7.0 (m, 2H), 7.1 (m, 2H) ppm.

### Example 58

(±)-trans-2-Ethoxy-3-[4-(4-fluoro-phenyl)-piperidin-3-ylmethoxy]-pyridine fumaric acid salt. To a solution of 2-ethoxy-pyridin-3-ol (0.12 g, 0.85 mmol) in dimethylformamide (5 mL) stirred at room temperature was added sodium hydride (60% by weight, 0.036 g, 0.91 mmol) in several portions. The mixture was stirred for 10 minutes followed by the addition of Intermediate 12 (0.32 g, 0.83 mmol) in a single portion. The solution was refluxed for overnight, cooled to room temperature, and diluted with 15 mL each of water and ethyl acetate. The organic phase was separated, dried over anhydrous sodium sulfate, filtered and the filtrate was concentrated in vacuo. Trans-3-(2-ethoxy-pyridin-3-yloxymethyl)-4-(4-fluoro-phenyl)-piperidine-1-carboxylic acid tert-butyl ester was isolated as a viscous liquid (0.28 g, 79%) using silica gel chromatography (elution with 70% hexane/30% ethyl acetate).

The trans-3-(2-ethoxy-pyridin-3-yloxymethyl )-4-(4-fluoro-phenyl)-piperidine-1-carboxylic acid tert-butyl ester (0.28 g, 0.65 mmol) was taken up in dichloromethane (10 mL) followed by the dropwise addition of trifluoroacetic acid (3 mL) at room temperature. The solution was stirred at room temperature for overnight, then concentrated in vacuo. The product was partitioned between saturated sodium bicarbonate and ethyl acetate (5 mL each). The organic phase was separated, dried over anhydrous sodium sulfate, filtered and concentrated to dryness. The resulting viscous liquid was taken up in 3 mL of anhydrous acetone and added dropwise to a solution of fumaric acid in acetone (0.075 g, 0.65 mmol in 5 mL acetone). The solution was stirred at room temperature for overnight affording a white precipitate. A solid was collected by filtration and dried in vacuo. Yield: 0.11 g, 38%.¹ H NMR (DMSO-d₆) δ 1.3 (t, 3H), 1.8 (d, 1 H), 1.9 (q, 1 H), 2.4 (m, 1 H), 2.7-2.9 (m), 3.3 (d, 1 H), 3.5 (d, 1 H), 3.6 (dd, 1 H), 3.7 (d, 1 H), 4.3 (t, 2H), 6.5 (s, 2H), 6.8 (m, 1 H), 7.0 (d, 1 H), 7.1 (t, 2H), 7.3 (m, 2H), 7.6 (d,1 H) ppm. C₁₉H₂₃FN₂O₂ 1.0 C₄H₄O₄: %C_{calcd.} 61.87 %C_{found} 61.74; %H_{calcd.} 6.10 %H_{found} 6.08; %N_{calcd.} 6.27 %N_{found} 6.24.

The title compound of Example 59 was synthesized in a manner similar to Example 58 by replacing the (±)-trans-4-(4-fluoro-phenyl)-piperidine-1,3-dicarboxylic acid-1-tert-butyl ester 3-ethyl ester used in the synthesis of Intermediate 10 with (±)-trans-4-(3-fluoro-phenyl)-piperidine-1,3-dicarboxylic acid-1-tert-butyl ester 3-ethyl ester:

### Example 59

(±)-trans-2-Ethoxy-3-[4-(3-fluoro-phenyl)-piperidin-3-ylmethoxy]-pyridine fumaric acid salt. ¹ H NMR (DMSO-d₆)) δ 1.3 (t, 3H), 1.8 (d, 1 H), 1.9 (q, 1 H), 2.4 (m, 1 H), 2.8 (m, 3H), 3.3 (d, 1 H), 3.2 (d, 1 H), 3.5 (d, 1 H), 3.6 (m, 2H), 3.7 (d, 1 H), 4.3 (q, 2H), 6.4 (s, 2H), 6.8 (m, 1 H), 7.0 (d, 1 H), 7.1 (t, 2H), 7.3 (m, 2H), 7.6 (d, 1 H) ppm. Microanalysis: C₁₉H₂₃FN₂O₂ 1.0 C₄H₄O₄: %C_{calcd.} 61.87 %C_{found} 61.51; %H_{calcd.} 6.10 %H_{found} 6.04; %N_{calcd.} 6.27 %N_{found} 6.18.

The title compounds of Examples 60 to 63 were synthesized in a manner similar to Example 58 by replacing the (±)-trans- 4-(4-fluoro-phenyl)-piperidine-1,3-dicarboxylic acid-1-tert-butyl ester 3-ethyl ester used in the synthesis of Intermediate 10 with (±)-trans-4-phenylpiperidine-1,3-dicarboxylic acid-1-tert-butyl ester 3-ethyl ester, and replacing the 2-ethoxy-pyridin-3-ol used in the synthesis of Example 58 with the appropriately substituted pyridinol. In addition, the hydrolysis of the butoxycarbonyl ester was carried out using hydrochloric acid in diethyl ether instead of using fumaric acid in acetone.

### Example 60

(±)-trans-2-Ethyl-3-(4-phenyl-piperidin-3-ylmethoxy)-pyridine hydrochloride. ¹ H NMR (DMSO-d₆) δ 1.2 (t, 3H), 1.8 (d, 1 H), 2.1 (q, 1 H), 2.7 (m, 1 H), 2.8-3.0 (m, 4H), 3.3 (d, 1 H), 3.5 (d, 1 H), 3.7 (m, 1 H), 3.9 (d, 1 H), 7.2 (m, 3H), 7.3 (m, 2H), 7.7 (m, 1 H), 7.8 (d, 1 H), 8.3 (d, 1 H), 9.4 (bs, 1 H), 9.6 (bs, 1 H) ppm. Microanalysis: C₁₉H₂₄N₂O 1.9 HCl/1.8 H₂O: %C_{calcd.} 59.19 %C_{found} 59.27; %H_{calcd.} 7.92 %H_{found} 8.07; %N_{calcd.} 6.57 %N_{found} 6.52.

### Example 61

(±)-trans-2-Butyl-3-(4-phenyl-piperidin-3-ylmethoxy)-pyridine hydrochloride. ¹ H NMR (DMSO-d₆) δ 0.9 (t, 3H), 1.3 (m, 2H), 1.6 (m, 2H), 1.9 (d, 1 H), 2.1 (q, 1 H), 2.7 (m, 1 H), 2.8-3.0 (m, 4H), 3.4 (d, 1 H), 3.5 (d, 1 H), 3.8 (m, 1 H), 3.9 (d, 1 H), 7.2 (m, 3H), 7.3 (m, 2H), 8.2 (d, 1 H), 9.4, (m, 1 H), 9.5 (m, 1 H) ppm. Microanalysis: C₂₁ H₂₈N₂O 1.9 HCl/2.6 H₂O: %C_{calcd.} 57.25 %C_{found}57.42; %H_{calcd.}8.03 %H_{found} 8.04; %N_{calcd.} 6.36 %N_{found} 6.12.

### Example 62

(±)-trans-2-lsobutyl-3-(4-phenyl-piperidin-3-ylmethoxy)-pyridine hydrochloride. ¹ H NMR (CDCl₃) δ 0.9 (s, 6H), 2.0 (d, 1 H), 2.2 (bs, 1 H), 2.4 (m, 1 H), 2.7 (bs, 1 H), 3.0 (bs, 4H), 3.6-4.0 (m, 5H), 7.1 (m, 5H), 7.2-7.4 (bs, 2H), 8.2 (s, 1 H), 9.8 (bs, 1 H), 10.4 (bs, 1 H) ppm. Microanalysis: C₂₁ H₂₈N₂O 1.9 HCl/2.6 H₂0: %C_{calcd.} 57.25 %C_{found} 57.42; %H_{calcd.} 8.03 %H_{found} 8.04; %N_{calcd.} 6.36 %N_{found} 6.12.

### Example 63

(±)-trans-2-Cyclopentyl-3-(4-phenyl-piperidin-3-ylmethoxy)-pyridine hydrochloride. ¹ H NMR (DMSO-d₆) δ 1.7 (s, 2H), 1.8 (s, 4H), 1.8-2.2 (m, 2H), 2.7 (m, 1 H), 2.8-3.0 (m, 2H), 3.4, (d, 1 H), 3.5 (m, 2H), 3.7-3.9 (m, 2H), 7.2 (m, 3H), 7.3 (m, 2H), 7.6 (bs, 1 H), 7.7 (bs, 1 H), 8.2 (s, 1 H), 9.4 (bs, 1 H), 9.6 (bs 1 H) ppm. Microanalysis: C₂₂H₂₇N₂O 1.9 HCl/2.6 H₂O: %C_{calcd.} 58.51 %C_{found} 58.47; %H_{calcd.} 7.61 %H_{found} 7.55; %N_{calcd.} 6.20 %N_{found} 6.03.

### BIOLOGICAL EXAMPLE 1

hNET Receptor Binding:

Cell pastes of HEK-293 cells transfected with a human norepinephrine transporter cDNA were prepared. The cell pastes were resuspended in 400 to 700 mL of Krebs-HEPES assay buffer (25 mM HEPES, 122 mM NaCl, 3 mM KCl, 1.2 mM MgSO₄, 1.3 mM CaCl₂, and 11 mM glucose, pH 7.4) with a Polytron homogenizer at setting 7 for 30 seconds. Aliquots of membranes (5 mg/mL protein) were stored in liquid nitrogen until used.

The binding assay was set up in Beckman deep-well polypropylene plates with a total volume of 250 μL containing: compound of one of the Examples (10⁻⁵M to 10⁻¹²M), cell membranes, and 50 pM [¹²⁵I]-RTI-55 (Perkin Elmer, NEX-272; specific activity 2200 Ci/mmol). The reaction was incubated by gentle agitation for 90 minutes at room temperature and was terminated by filtration through Whatman GF/C filter plates using a Brandel 96-well plate harvester. Scintillation fluid (100 μL) was added to each well, and bound [¹²⁵I]-RTI-55 was determined using a Wallac Trilux Beta Plate Counter. Test compounds were run in duplicate, and specific binding was defined as the difference between binding in the presence and absence of 10 μM desipramine.

Excel and GraphPad Prism software were used for data calculation and analysis. IC₅₀ values were converted to Kᵢ values using the Cheng-Prusoff equation. The Kᵢ values (nM) for the hNET for the compounds of the specified Examples are reported below in Table 1.

hSERT Receptor Binding:

Cell pastes of HEK-293 cells transfected with a human serotonin transporter cDNA were prepared. The cell pastes were resuspended in 400 to 700 mL of Krebs-HEPES assay buffer (25 mM HEPES, 122 mM NaCl, 3 mM KCl, 1.2 mM MgSO₄, 1.3 mM CaCl₂, and 11 mM glucose, pH 7.4) with a Polytron homogenizer at Setting 7 for 30 seconds. Aliquots of membranes (about 2.5 mg/mL protein) were stored in liquid nitrogen until used.

Assays were set up in FlashPlates pre-coated with 0.1 % PEI in a total volume of 250 μL containing: compound of one of the Examples (10⁻⁵M to 10⁻¹²M), cell membanes, and 50 pM [¹²⁵l]-RTI-55 (Perkin Elmer, NEX-272; specific activity 2200 Ci/mmol). The reaction was incubated and gently agitated for 90 minutes at room temperature, and terminated by removal of assay volume. Plates were covered, and bound [¹²⁵I]-RTI-55 was determined using a Wallac Trilux Beta Plate Counter. Test compounds were run in duplicate, and specific binding was defined as the difference between binding in the presence and absence of 10 μM citalopram.

Excel and GraphPad Prism software were used for data calculation and analysis. IC₅₀ values were converted to Kᵢ values using the Cheng-Prusoff equation. The Kᵢ values (nM) for hSERT for the compounds of the specified Examples are reported below in Table 1

TABLE 1

| | NET Kᵢ | SERT Kᵢ |
|---|---|---|
| Ex. No. | (nM) | (nM) |
| 1 | 3.831 | 24.26 |
| 2 | 3289 | 6.507 |
| 3 | 1262 | 18.81 |
| 4 | 1871 | 15.26 |
| 5 | 2621 | 2.29 |
| 6 | 3475 | 0.5202 |
| 7 | 3.341 | 17.68 |
| 8 | 1332 | 16.97 |
| 9 | 3.04 | 17.48 |
| 10 | 6258 | 18.55 |
| 11 | 4.57 | 188.4 |
| 12 | 4.18 | 40.58 |
| 13 | 612.1 | 2.69 |
| 14 | 12.06 | 163.7 |
| 15 | 4.9 | 20.75 |
| 16 | 293.5 | 679.9 |
| 17 | 13.21 | 21.88 |
| 18 | 1.75 | 5.37 |
| 19 | 144.2 | 29.1 |
| 20 | 156.9 | 3.14 |
| 21 | 1262 | 63.78 |
| 22 | 263.3 | 3.47 |
| 23 | 1520.05 | 1386.11 |
| 24 | 518.61 | 87.67 |
| 25 | 107.25 | 2.49 |
| 26 | 73.31 | 21.48 |
| 27 | 10000 | 5228.27 |
| 28 | 6379.78 | 2128.49 |
| 29 | 309.3 | 966.84 |
| 30 | 534.03 | 333.32 |
| 31 | 101.66 | 889.92 |
| 32 | 2939.20 | 861.31 |
| 33 | 8.60 | 18.06 |
| 34 | 1439.41 | 3500.08 |
| 35 | 14.55 | 8.4 |
| 36 | 101.18 | 13.67 |
| 37 | 1025.70 | 430.60 |
| 38 | 12.10 | 173.55 |
| 39 | 145.88 | 731.86 |
| 40 | 750.75 | 5676.01 |
| 41 | 3.88 | 2.67 |
| 42 | 1.7 | 626 |
| 43 | 14.41 | 89.69 |
| 44 | 33.25 | 158.07 |
| 45 | 36.47 | 71.83 |
| 46 | 232.67 | 156.46 |
| 47 | 51.50 | 1051.50 |
| 48 | 2.10 | 22.33 |
| 49 | 19.50 | 83.50 |
| 50 | 16.00 | 33.00 |
| 51 | 75.00 | 174.00 |
| 52 | 35.00 | 96.00 |
| 53 | 138.50 | 267.00 |
| 54 | 36.50 | 208.00 |
| 55 | 215.50 | 1051.50 |
| 56 | 317.00 | 542.50 |
| 57 | 192.70 | 34.78 |
| 58 | 6.25 | 5.97 |
| 59 | 4.38 | 10.67 |
| 61 | 7.48 | 23.81 |
| 62 | 46.55 | 59.25 |

### BIOLOGICAL EXAMPLE 2

Compounds of the present invention may be assayed for their ability to alleviate capsaicin-induced mechanical allodynia in a rat (e.g., Sluka (2002) J of Neuroscience, 22(13): 5687-5693). For example, a rat model of capsaicin-induced mechanical allodynia was carried out as follows:

On day 0, male Sprague-Dawley rats (about 150 g each) in the dark cycle were placed in suspended wire-bottom cages and allowed to acclimate for 0.5 hour in a darkened, quiet room. The day 0 paw withdrawal threshold (PWT) was determined on the left hind paw by Von Frey hair assessment using the Dixon up and down method. After assessment, the plantar muscle of the right hind paw was injected with 100 μL capsaicin (0.25% (w/v) in 10% ethanol, 10% Tween 80, in sterile saline). On day 6 the PWT of the left hind paw (contralateral from injection site) was determined for each animal. Animals from the day 6 prereads with PWT ≦11.7 g were considered allodynic responders and were regrouped so that each cage had similar mean PWT values. On day 7, responders were dosed subcutaneously with 10 mg compound/kg body weight, or with vehicle alone. The vehicle was phosphate buffered saline containing 2% Cremophor® EL (BASF Corp., Florham Park, N.J.). The contralateral PWT values were determined at 1 hour after the single dose, with the investigator blinded to the dosing scheme.

For each animal, the day 6 PWT value was subtracted from the 1 hour PWT value to give a delta PWT value that represents the change in PWT due to the 1 hour drug treatment. In addition, the day 6 PWT was subtracted from the day 0 PWT to give the baseline window of allodynia present in each animal. To determine % inhibition of allodynia of each animal normalized for vehicle controls, the following formula was used: % Inhibition of Allodynia =100× [(Delta PWT(drug)—mean Delta PWT(vehicle))/(Baseline—mean Delta PWT(vehicle))].

The mean percent inhibition of allodynia values (for eight animals assayed for each compound) +/− the standard error of the mean (SEM) are shown in Table 2. Compounds exhibiting a greater than 30% inhibition in allodynia assay (and are statistically significant compared to vehicle by ANOVA/Dunnett's tests) are considered active.

TABLE 2

| Example Number | % Inhibition |
|---|---|
| 1 | 82.3 +/− 9.3 |
| 2 | 1.4 +/− 4.7 |
| 7 | 67.3 +/− 15.7 |
| 9 | 29.5 +/− 7.8 |
| 11 | 77.3 +/− 10.8 |
| 12 | 66.4 +/− 14.3 |
| 14 | 22.7 +/− 4.3 |
| 17 | 2.6 +/− 2.6 |
| 35 | 5.5 +/− 5.1 |
| 38 | 0.2 +/− 5.2 |

It is understood that the examples and embodiments described herein are for illustrative purposes only and that various modifications or changes in light thereof will be suggested to persons skilled in the art and are to be included within the spirit and purview of this application and the scope of the appended claims. All publications, patents, and Pat. applications cited herein are hereby incorporated by reference in their entirety for all purposes.

What is claimed is:

1. A compound of Formula I: 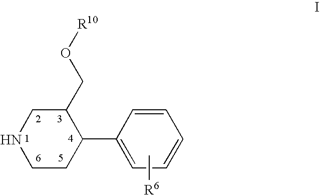
or a pharmaceutically acceptable salt thereof, wherein: the positions of the piperidinyl group of Formula I are labeled as 1, 2, 3, 4, 5, or 6; R¹⁰ is 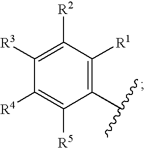
a thienopyridinyl, a 5-membered heteroaryl, or a 6-membered heteroaryl;
R¹, R², R³, R⁴, and R⁵ are independently selected from the group consisting of: H;
—O—C₅-C₇-cycloalkyl; C₅-C₇-cycloalkyl; —O—C₅-C₇-heterocycloal kyl;
-C₅-C₇-heterocycloalkyl; —O—phenyl; phenyl; C₁₋₄alkylene-NR¹⁶R¹⁸; C₁₋₄alkyl;
C₁₋₄alkoxy; halo; —C(O)NR¹²R¹⁴; —SO₂—CH₃; —SO₂—CH₂CH₃; and CN;
wherein said thienopyridinyl, 5-membered heteroaryl or 6-membered heteroaryl may be
optionally substituted with one to three substituents independently selected from the group consisting of: H; —O—C₅-C₇-cycloalkyl; C₅-C₇-cycloalkyl;
—O—C₅-C₇-heterocycloalkyl; -C₅-C₇-heterocycloalkyl; —O—phenyl;
—O—(CH₂)ₙ-phenyl; -(CH₂)n-phenyl; phenyl; —O—(5- or 6-membered heteroaryl);
—O—(CH₂)ₙ-(5- or 6-membered heteroaryl); -(CH₂)ₙ-(5- or 6-membered heteroaryl); C₁₋₄alkylene-NR¹⁶R¹⁸; C₁₋₄alkyl; C₁₋₄alkoxy; halo;
—C(O)NR¹²R¹⁴; —SO₂—CH₃; —SO₂—CH₂CH₃; and CN;
n is 1,2, or3;
R¹² and R¹⁴ are independently selected from the group consisting of: H and C₁₋₄alkyl;
R¹⁶ and R¹⁸ are independently selected from the group consisting of: H and C₁₋₄alkyl; and
R⁶ is H or one to three substituents independently selected from the group consisting of:
C₁₋₄alkyl; C₁₋₄alkoxy; and halo;
provided that when R¹⁰ is 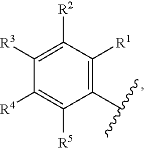
R¹, R², R⁴, and R⁵ are H, then R³ is not C₁₋₄alkoxy; and
when R¹⁰ is 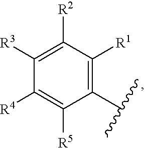
then one of R¹, R², R³, R⁴, R⁵, and R⁶ is not H.

2. The compound of claim 1, or a pharmaceutically acceptable salt thereof, wherein the 3-position of the piperidinyl group of Formula I is of the S conformation and the 4-position of the piperidinyl group of Formula I is of the R conformation.

3. The compound of claim 2, or a pharmaceutically acceptable salt thereof, wherein R¹⁰ is 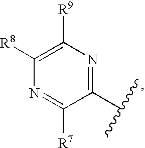
thieno[3,2-b]pyridin-7-yl, 1 H-pyrazol-3-yl, or 2H-pyrazol-3-yl, wherein R⁷, R⁸, and R⁹ are independently selected from the group consisting of: H; —O—C₅-C₇-cycloalkyl; C₅-C₇-cycloalkyl; —O—C₅-C₇-heterocycloalkyl; -C₅-C₇-heterocycloalkyl; 13 O—phenyl; phenyl; C₁₋₄alkylene-NR¹⁶R¹⁸; C₁₋₄alkyl; C₁₋₄alkoxy; halo; —C(O)NR¹²R¹⁴; —SO₂—CH₃; —SO₂—CH₂CH₃; and CN.

4. The compound of claim 2, or a pharmaceutically acceptable salt thereof, wherein R¹⁰ is 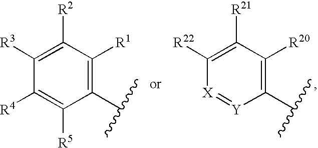
wherein
X is C(R²³) and Y is N, or X is N and Y is C(R²⁴); and R²⁰, R²¹, R²², R²³, and R²⁴ are independently selected from the group consisting of: H;
—O—C₅-C₇-cycloa Ikyl; C₅-C₇-cycloalkyl; —O—C₅-C₇-heterocycloal kyl;
—C₅-C₇-heterocycloalkyl; —O—phenyl; phenyl; C₁₋₄alkylene-NR¹⁶R¹⁸; C₁₋₄alkyl;
C₁₋₄alkoxy; halo; -C(O)NR¹²R¹⁴; —SO₂—CH₃; —SO₂—CH₂CH₃; and CN.

5. The compound of claim 4, or a pharmaceutically acceptable salt thereof, wherein
R¹⁰ is 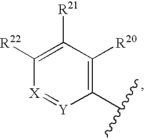
X is C(R²³) and Y is N.

6. The compound of claim 4, or a pharmaceutically acceptable salt thereof, wherein R¹⁰ is

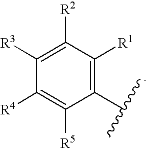

7. The compound of claim 6, or a pharmaceutically acceptable salt thereof, wherein R¹, R², R³, R⁴, and R⁵ are independently selected from the group consisting of:
H; C₁₋₄alkyl; C₁₋₄alkoxy; halo; -C(O)NR¹²R¹⁴; —SO₂—CH₃; —SO₂—CH₂CH₃; and CN.

8. The compound of claim 4, or a pharmaceutically acceptable salt thereof, wherein R¹⁰ is 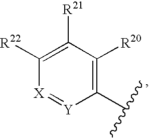
wherein X is N and Y is C(R²⁴).

9. The compound of claim 8, or a pharmaceutically acceptable salt thereof, wherein R²⁰, R²¹, R²², and R²⁴ are independently selected from the group consisting of:
H; C₁₋₄alkyl; C₁₋₄alkoxy; halo; -C(O)NR¹²R¹⁴; —SO₂—CH₃; —SO₂—CH₂CH₃; and CN.

10. The compound of claim 1, wherein the compound is selected from the group consisting of:
(3S,4R)-7-(4-phenyl-piperidin-3-ylmethoxy)-thieno[3,2-b]pyridine;
(3S,4R)-3-(2-ethoxy-phenoxymethyl)-4-phenyl-piperidine;
(3S, 4R)-2-(4-phenyl-piperidin-3-ylmethoxy)-benzon itrile;
(3S,4R)-3-(2-fluoro-6-methoxy-phenoxymethyl)-4-phenyl-piperidine;
(3S,4R)-2-ethoxy-3-(4-phenyl-piperidin-3-ylmethoxy)-pyridine;
(3S,4R)-2-methoxy-3-(4-phenyl-piperidin-3-ylmethoxy)-pyridine;
(3S,4R)-3-(4-phenyl-piperidin-3-ylmethoxy)-2-propoxy-pyridine;
(3S,4R)-2-(2-methoxy-ethoxy)-3-(4-phenyl-piperidin-3-ylmethoxy)-pyridine;
(3S,4R)-2-isopropoxy-3-(4-phenyl-piperidin-3-ylmethoxy)-pyridine;
(3S,4R)-2-(3-methoxy-propoxy)-3-(4-phenyl-piperidin-3-ylmethoxy)-pyridine;
(3S,4R)-2-isobutoxy-3-(4-phenyl-piperidin-3-ylmethoxy)-pyridine; and
(±)-trans-2-ethoxy-3-[4-(4-fluoro-phenyl)-piperidin-3-ylmethoxy]-pyridine;
or a pharmaceutically acceptable salt thereof.

11. A pharmaceutical composition comprising:
a therapeutically effective amount of a compound or a pharmaceutically acceptable salt thereof according to claim 1 and a pharmaceutically acceptable excipient.

12. A method of treating a disorder or condition selected from the group consisting of:
fibromyalgia; attention deficit hyperactivity disorder; generalized anxiety disorder;
depression; and schizophrenia, the method comprising administering to a mammal in need of such treatment a therapeutically effective amount of a compound or a pharmaceutically acceptable salt thereof according to claim 1.
